(19) United States
(12) Patent Application Publication
(10) Pub. No.: US 2025/0232838 A1
(43) Pub. Date: Jul. 17, 2025

(54) METHODS AND SYSTEMS FOR IDENTIFYING AND VALIDATING A GENE COMBINATION ASSOCIATED WITH A TRAIT AND USES THEREOF

(71) Applicant: Passkey Therapeutics, Inc., Cambridge, MA (US)

(72) Inventors: Bruce Aaron BEUTEL, Wakefield, MA (US); William W. CHEN, Winchester, MA (US); Finola MOORE, Cambridge, MA (US); Matthew BELMONTE, Hollis, NH (US); Valeria PADOVANO, Medford, MA (US); Nghia MILLARD, Boston, MA (US); Peter J. DANDLIKER, Quincy, MA (US)

(21) Appl. No.: 19/025,625

(22) Filed: Jan. 16, 2025

Related U.S. Application Data
(60) Provisional application No. 63/621,878, filed on Jan. 17, 2024.

(51) Int. Cl. G16B 20/20; C12N 9/22; C12N 15/11; C12N 15/113; G01N 33/50; G16B 35/20; G16B 40/30; G16H 50/20

(52) U.S. Cl. CPC G16B 20/20; C12N 9/22; C12N 15/111; C12N 15/113; G01N 33/5023; G16B 35/20; G16B 40/30; G16H 50/20; C12N 2310/14; C12N 2310/20

(57) ABSTRACT

The present application relates to methods and systems for identifying and validating duologs and multilogs (gene combinations), including derivatives thereof, e.g., a polypeptide, that are associated with a trait such as a human disease. In certain aspects, the methods and systems provided herein are useful for identifying synergistic drug targets for treating a condition, such as a disease, in an individual.

### CROSS-REFERENCE TO RELATED APPLICATIONS

This patent application claims priority benefit of U.S. Provisional Application No. 63/621,878, filed Jan. 17, 2024, the content of which is incorporated herein by reference in their entirety.

### FIELD

The present application relates to methods and systems for identifying and validating duologs and multilogs (gene combinations), including derivatives thereof, e.g., polypeptides, that are associated with a trait. In certain aspects, the methods and systems provided herein are useful for identifying synergistic drug targets for treating a condition, such as a disease, in an individual.

### BACKGROUND

Conventional drug discovery approaches are built on a foundation of identifying a single compound that specifically modulates a single target. Such monogenic drug discovery methods include identifying a gene and/or protein associated with a condition (e.g., a disease) and then screening candidate compounds based on modulation of said gene and/or protein to treat the condition. As our understanding of cellular mechanisms grows, it is evident that monogenic approaches to drug discovery are limited in their ability to: address more complex conditions caused by more than one gene (or derivatives thereof), and/or consider alternatives to treatments focused on a single target. There remains a need for approaches to identify underlying genes associated with polygenic traits, such as a human disease, as well as validating polygenic approaches capable of improved treatment and/or prevention of a human condition.

### BRIEF SUMMARY

In some aspects, provided herein are methods for identifying one or more gene combinations associated with a trait, the method comprising: identifying one or more genetic variant combinations from a genetic data set based on one or more variant interaction scores, wherein each variant interaction score is representative of an association between a trait and a joint state of all genetic variants of a single genetic loci combination of the one or more genetic loci combinations, wherein the genetic data set comprises a plurality of inputs each having genetic information and at least one label indicative of the trait; identifying one or more genes associated with genetic variants of the one or more identified genetic variant combinations; selecting gene groupings from a library based on each gene grouping containing at least one of the identified one or more genes to form one or more gene grouping sets, wherein the library comprises groupings each representing an independent aspect of biology; determining an interaction-density score for each of the one or more gene grouping sets wherein the interaction-density score is based on normalization of a grouping interaction score determined from the variant interaction scores for the genetic variant combinations between gene groupings in the gene grouping set; and identifying the one or more gene combinations associated with the trait from at least one of the one or more gene grouping sets selected based on the determined interaction-density scores.

In some aspects, the trait is the presence or absence of a human condition. In some aspects, the trait is a metric that is relevant to a human condition. In some aspects, the metric is assessed at the molecular, cellular, and/or organismal level. In some aspects, the human condition is a disease. In some aspects, the human disease is a metabolic disease, a cardiovascular disease, an immune disease, a neuro-degenerative disease, a non-neuro-degenerative disease, or a cancer.

In some aspects, genetic variant combinations contain genetic variants wherein each is independently selected from the group consisting of SNP, structural variation, copy number variation, insertion, deletion, translocation, and inversion.

In some aspects, the genetic data set comprises an input assembled from genotype chip calls and/or exome arrays. In some aspects, the genetic data set comprises an input from a published data set. In some aspects, the method further comprises assembling the genetic data set.

In some aspects, the genetic data set, or a portion thereof, is cleaned. In some aspects the genetic data set is cleaned by removing inputs outside a homogeneous population of inputs. In some aspects, the genetic data set is cleaned by removing inputs with missing genetic information above about 2%. In some aspects, the genetic data set is cleaned by removing genetic information with missing information for above about 2% of inputs. In some aspects, the genetic data set is cleaned by removing genetic information containing a variant below about 5% across the inputs. In some aspects, the methods further comprise cleaning at least one of the plurality of inputs of the genetic data set.

In some aspects, the genetic variant combinations are cleaned by removing genetic variant combinations that have a linkage disequilibrium correlation exceeding about 0.1, wherein the linkage disequilibrium correlation is computed on a control genotyped cohort. In some aspects, the method further comprises cleaning the genetic variant combinations.

In some aspects, the variant interaction score is the synergy between the trait and a combined state of the genetic variant combinations. In some aspects, the variant interaction score relates to a logistic or linear regression that models the state of each genetic variant in the genetic variant combination as a bivariate linear predictor. In some aspects, the methods herein further comprise transforming the variant interaction score. In some aspects, the transforming is done with a shifted Heaviside function or thresholding the values to exceed 0, whereby any negative values are converted to 0.

In some aspects, the variant interaction score relates to a decisions rule comprising more than one statistical tests. In some aspects, the one or more statistical tests comprises a statistical test selected from a group consisting of a hypergeometric test, a logistic regression, or a linear regression.

In some aspects, the identifying one or more genes associated with genetics variants of the identified plurality of genetic variant combinations is performed by inputting the genetic variants into a trained machine learning model configured to accept a genetic variant and output a gene. In some aspects, the machine learning model is trained using Hi-C, eQTL, pQTL, and genomic coordinates.

In some aspects, the library comprises gene groupings from one or more of Reactome, BioCarta, WikiPathways, Pathways Commons, HPRD, STRING/IntACT, PANTHER, ENCODE, or other groupings associated with a characteristic. In some aspects, the library comprising groupings each representing an independent aspect of biology comprises one or more gene networks. In some aspects, the library comprising groupings each representing an independent aspect of biology comprises gene groupings where each gene grouping has at least one non-overlapping gene when compared directly to another gene grouping in the library. In some aspects, the methods further comprise assembling the library.

In some aspects, the grouping interaction score is a based on the variant interaction scores for the genetic variant combinations between the gene groupings in the gene grouping set. In some aspects, the group interaction score is normalized by dividing the score by the theoretical maximum of the number of genetic variant combinations between gene groupings in the gene grouping set.

In some aspects, the gene grouping sets are filtered out of the method if the interaction density score is below a computed background distribution. In some aspects, the computed background distribution is related to a permuted distribution of shuffled genetic variants combinations.

In some aspects, the gene combinations show gene expression differences in a trait relevant cell type or tissue.

In some aspects, the identified gene combinations are selected from gene grouping scores with an interaction-density score above the interaction-density scores in the computed background distribution.

In some aspects, the methods comprise experimentally validating one or more of the identified gene combinations in a disease model system. In some aspects, an identified gene combination is validated based on an observed phenotype consistent with a phenotype that may treat or prevent a human condition. In some aspects, wherein the phenotype comprises a characteristic based on one or more of cell growth inhibition, pro- or anti-apoptotic activity, inhibition or stimulation of a cellular stress response, modulation of glucose metabolism, modulation of insulin-dependent metabolism, or production or inhibition of disease-related polypeptides. In some aspects, the disease model system comprises a cell assay, an organoid assay, or an animal model.

In some aspects, the experimental validation comprises modulating an activity and/or expression level of an identified gene combination and comparing that to modulating an activity and/or expression level of one or more genes of the identified gene combination. In some aspects, the experimental validation comprises a gene knockdown or knockout technique. In some aspects, the gene knockdown or knockout technique is a siRNA technique or a CRISPR technique.

In some aspects, the methods further comprise selecting at least one of the identified gene combinations based on co-druggability.

In some aspects, the methods further comprising experimentally validating the identified one or more gene combinations associated with the trait, wherein the identified one or more gene combinations comprises a gene combination comprising a first gene and a second gene. In some aspects, the experimental validating comprises performing a cell-based assay on: (i) a first cell sample subjected to a programmable genome or transcriptome modulator to modulate the expression of the first gene; (ii) a second cell sample subjected to a programmable genome or transcriptome modulator to modulate the expression of the second gene; and (iii) a third cell sample subjected to the programmable genome or transcriptome modulator to modulate the expression of the first gene and the programmable genome or transcriptome modulator to modulate the expression of the second gene, and determining if modulation of the expression of the first gene and the second gene results in a synergistic response observed in the cell-based assay. In some aspects, the first cell sample and the second cell sample are further subjected to a non-targeting programmable genome or transcriptome modulator control. In some aspects, the programmable genome or transcriptome modulator for the first gene and the programmable genome or transcriptome modulator for the second gene are each selected from the group consisting of RNAi, a TALE-based modulation system, a zinc-finger-based modulation system, a meganuclease-based editing system, an epigenomic-based editing system, a mRNA editing system, and a CRISPR-based modulation system. In some aspects, the programmable genome or transcriptome modulator for the first gene and the programmable genome or transcriptome modulator for the second gene are each an RNAi. In some aspects, the RNAi is siRNA.

In some aspects, the experimental validating comprises performing a cell-based assay on: (i) a first cell sample subjected to a drug moiety modulating the first gene, or an expression product thereof, and a non-target programmable genome or transcriptome modulator; and (ii) a second cell sample subjected to a drug moiety modulating the first gene and a programmable genome or transcriptome modulator targeting the second gene, and determining if there is an improved result in the cell-based assay from the second cell sample as compared to the first cell sample. In some aspects, the drug moiety is a small molecule compound, peptide, protein, or antibody. In some aspects, the drug moiety is an inhibitor. In some aspects, the drug moiety is an activator. In some aspects, each programmable genome or transcriptome modulator is selected from the group consisting of RNAi, a TALE-based modulation system, a zinc-finger-based modulation system, a meganuclease-based editing system, an epigenomic-based editing system, a mRNA editing system, and a CRISPR-based modulation system. In some aspects, the cell-based assay is selected from the group consisting of a cell viability assay, a cell growth assay, a cell proliferation assay, a growth inhibition assay, and a metabolic assay.

Also provided herein are methods of experimentally validating a gene combination comprising, the method comprising performing a cell-based assay on: (i) a first cell sample subjected to a drug moiety modulating the first gene, or an expression product thereof, and a non-target programmable genome or transcriptome modulator; and (ii) a second cell sample subjected to a drug moiety modulating the first gene and a programmable genome or transcriptome modulator targeting the second gene, and determining if there is an improved result in the cell-based assay from the second cell sample as compared to the first cell sample. In some aspects, the gene combination is identified using a method according to any of the methods for identifying one or more gene combinations associated with a trait described herein.

In some aspects, provided herein are systems for identifying one or more gene combinations associated with a trait, the system comprising, one or more processors; and memory storing one or more programs, the one or more programs configured to be executed by the one or more processors, the one or more programs including instructions for: identifying one or more genetic variant combinations from a genetic data set based on one or more variant interaction scores, wherein each variant interaction score is representative of an association between a trait and a joint state of all genetic variants of a single genetic loci combination of the one or more genetic loci combinations, wherein the genetic data set comprises a plurality of inputs each having genetic information and at least one label indicative of the trait; identifying one or more genes associated with genetic variants of the one or more identified genetic variant combinations; selecting gene groupings from a library based on each gene grouping containing at least one of the identified one or more genes to form one or more gene grouping sets, wherein the library comprises groupings each representing an independent aspect of biology; determining an interaction-density score for each of the one or more gene grouping sets wherein the interaction-density score is based on normalization of a grouping interaction score determined from the variant interaction scores for the genetic variant combinations between gene groupings in the gene grouping set; and identifying the one or more gene combinations associated with the trait from at least one of the one or more gene grouping sets selected based on the determined interaction-density scores.

In some aspects, provided herein are methods for identifying a subpopulation of patients for drug response, the method comprising: identifying one or more genetic variant combinations from a genetic data set based on one or more variant interaction scores, wherein each variant interaction score is representative of an association between a trait targeted by a drug and a joint state of all genetic variants of a single genetic loci combination of the one or more genetic loci combinations, wherein the genetic data set comprises a plurality of inputs each having genetic information and at least one label indicative of the trait; identifying one or more genes associated with genetic variants of the one or more identified genetic variant combinations; selecting gene groupings from a library based on each gene grouping containing at least one of the identified one or more genes to form one or more gene grouping sets, wherein the library comprises groupings each representing an independent aspect of biology; determining an interaction-density score for each of the one or more gene grouping sets wherein the interaction-density score is based on normalization of a grouping interaction score determined from the variant interaction scores for the genetic variant combinations between gene groupings in the gene grouping set; and identifying the one or more gene combinations associated with the trait from at least one of the one or more gene grouping sets selected based on the determined interaction-density scores; wherein a gene in each of the one or more gene combinations is a target of the drug; identifying a subpopulation of patients with genetic variation in one or more genes in the identified one or more gene combinations.

### BRIEF DESCRIPTION OF THE DRAWINGS

FIG. 1A-1C shows an illustrative example workflow following a single genetic variant pair through a method described herein for identifying one or more gene combinations associated with a trait. Specifically, FIG. 1A is an example of generic genetic data. FIG. 1B shows an example gene library with genes in the generic genetic data represented in FIG. 1A. FIG. 1C shows the example workflow incorporating the generic genetic data in FIG. 1A and grouping libraries in FIG. 1B.

FIG. 2 shows an illustrative example workflow, including inputs and outputs of method steps, of a method described herein for identifying one or more gene combinations associated with a trait.

FIGS. 3A-3E show results based on the experimental validation of a primary screen of genes identified using the methods taught herein. Specifically, FIG. 3A-FIG. 3D show plots of the effect on cancer cell growth inhibition for tested gene knockdown pairs. FIG. 3A shows the effect on cancer cell inhibition knocking down P2RY14 and POL3D. FIG. 3B shows the effect on cancer cell inhibition knocking down P2RY14 and POLR2L. FIG. 3C shows the effect on cancer cell inhibition knocking down P2RY14 and LZTS1. FIG. 3D shows the effect on cancer cell inhibition knocking down P2RY14 and POLR2E. FIG. 3E shows a pathway analysis of top synergistic duologs identified in a particular pair of interacting groups of genes using the methods described herein.

FIGS. 4A and 4B show plots of percentage cell viability over various tested concentrations for the combination of a drug moiety inhibitor of target A and either a non-target siRNA or a siRNA targeting target B (FIG. 4A) and various tested concentrations for the combination of a drug moiety inhibitor of target B and either a non-target siRNA or a siRNA targeting target A (FIG. 4B).

FIG. 5A-5C shows the distributions of urinary albumin-to-creatine ratio (uACR) and estimated glomerular filtration rate (eGFR) in individuals with Type 2 diabetes (T2D) with (case) and without (control) renal, eye complications (retinopathy), and microvascular complications. FIG. 5A shows the distribution for renal complications. FIG. 5B shows the distribution for retinopathy complications. FIG. 5C shows the distribution for microvascular complications.

FIGS. 6A and 6B show pathway analyses for top gene grouping sets. FIG. 6A shows the relationship between genes in Network 1 and Network 2. FIG. 6B shows the relationship between genes in Network 3 and Network 4. The connection and strength of the connection between the genes relate to variant interaction scores for SNPs associated with the genes.

FIGS. 7A and 7B show plots of normalized cell viability for cells subjected to a high glucose treatment and treated with siRNAs for genes in a predicted duolog. FIG. 7A shows normalized cell viability for four replicates of cells treated with an siRNA targeting Network 2-Gene F, Network 1-Gene P or both genes in high glucose conditions. FIG. 7B shows normalized cell viability for three replicates of cells treated with an siRNA targeting Network 3-Gene J, Network 4-Gene H or both genes in high glucose conditions.

FIGS. 8A and 8B show secreted MCP-1 concentrations (inflammatory marker) and cell viability for cells treated with non-target siRNAs (siNT) and siRNAs for two duolog gene pairs (siNetwork 1-Gene P+siNetwork 2-Gene F and siNetwork 3-Gene J+siNetwork 4-Gene H) in normal glucose (NG) and high glucose (HG) conditions. FIG. 8A shows supernatant MCP-1 concentrations for cells treated with siNT, siNetwork 1-Gene P+siNetwork 2-Gene F, and siNetwork 3-Gene J+siNetwork 4-Gene H at NG and HG conditions. FIG. 8B shows cell viability measurements for cells treated with siNT, siNetwork 1-Gene P+siNetwork 2-Gene F, and siNetwork 3-Gene J+siNetwork 4-Gene H at NG and HG conditions.

### DETAILED DESCRIPTION

The present application provides, in certain aspects, methods and systems for identifying duologs and multilogs (described herein as gene combinations) associated with a trait, such as a human disease. A duolog refers to a gene combination consisting of two genes or expression products thereof and a multilog refers to a gene combination consisting of more than two genes or expression products thereof. Duologs and multilogs are novel drug targets for treating a disease.

The disclosure provides methods and systems for identifying gene combinations associated with a trait that in turn can be used, e.g., as drug targets for achieving a synergistic effect on said trait or a condition associated therewith. At the time of filing the instant application, there remains a need for advanced techniques to understand polygenic traits which cannot otherwise be explained or targeted based on conventional monogenic approaches. As demonstrated herein, the taught methods and systems provide for the identification of previously unappreciated gene combinations from different gene groupings, such as different pathways or networks, that are involved with a disease, such as pancreatic cancer. Furthermore, experimental validation techniques were shown that biologically characterize the said identified duologs and multilogs (gene combinations as described herein) to provide better understanding of the effect on the said trait and provide druggable targets for treatment. In certain embodiments described herein, said experimental validation is based on modulating each member of a gene combination using a unique programmable genome or transcriptome modulator, such as siRNA. In certain other embodiments described herein, said experimental validation incorporates a drug moiety modulator, such as a small molecule, peptide, or antibody to better assess the druggability of gene combinations identified herein. Accordingly, the methods described herein to discover and validate duologs and multilogs provide a much needed polygenic approach to understanding and treating complex traits.

It is noted that the methodology provided herein is capable of identifying useful duologs and multilogs (gene combinations as described herein) comprising genes that do not themselves harbor genetic variants associated with a trait. Such technology enables one to identify genes (and derivatives thereof, such as expression products, e.g., RNA and polypeptides) as potential drug targets that are not otherwise identifiable using techniques focused on variants or genes comprising the variants.

The methods and systems taught herein enable the systematic identification of duologs and multilogs (gene combinations as described herein) from different groupings of genes, such as networks, that together impact a trait. Gene groupings, such as gene networks with non-overlapping genes, likely represent different aspects of biology that previously would not have been identified as both relevant to the biology of the trait using conventional methods in the art, e.g. GWAS, especially not in combination. The groups of genes in a gene grouping have been curated based on cellular and/or molecular biology. In a non-limiting example, genes in a gene grouping may represent genes in the same biological pathway that has been verified through years of laboratory experimentation. The methods described herein, also aim to identify relationships between groups to undercover genes that may be related to each other in the biology of a trait but previously were considered to be independent because of their inclusion in different networks or pathways.

Using the inventions disclosed herein, one can prioritize genes from networks or pathways whose interactions contribute to a trait, identify gene combinations associated with a trait, and validate gene combinations as having synergistic effects on a trait as compared to individual genes of the combination. The inventions disclosed herein, can be used to identify, with high confidence, gene combinations (or derivatives thereof) that can serve as drug targets. Such methods and systems are an advancement in the field and are amenable to uses of certain data structures capable of reducing needed computing power. For example, in some embodiments, the methods provide technical advantages by organizing data in one or more knowledge graphs (KGs). In some embodiments, the one or more KGs may be layered together to increase efficiency of the methods disclosed therein. In some embodiments, the methods disclose include storing data in KGs as disclosed herein.

Thus, in some aspects, provided herein is a method for identifying one or more gene combinations associated with a trait, the method comprising: identifying one or more genetic variant combinations from a genetic data set based on one or more variant interaction scores, wherein each variant interaction score is representative of an association between all genetic variants of a single genetic variant combination of the one or more genetic variant combinations and a shared trait, wherein the genetic data set comprises a plurality of inputs each having genetic information and at least one label indicative of the trait; identifying one or more genes associated with genetic variants of the one or more identified genetic variant combinations; selecting gene groupings from a library based on each gene grouping containing at least one of the identified one or more genes to form one or more gene grouping sets, wherein the library comprises groupings each representing an independent aspect of biology; determining an interaction-density score for each of the one or more gene grouping sets wherein the interaction-density score is based on normalization of a grouping interaction score determined from the variant interaction scores for the genetic variant combinations between gene groupings in the gene grouping set; and identifying one of more gene combinations associated with the trait from at least one of the one or more gene grouping sets selected based on the determined interaction-density scores. In some aspects, the synergistic effect of manipulating the one or more gene combinations for a trait can be validated using genetic and pharmacogenetic techniques.

In some aspects, the methods described herein can be used iteratively to increase the precision of the methods. For example, as described herein, the results of the validation experiments can also be used to further identify gene grouping sets that likely contain multiple gene combinations that can be drug discovery targets.

In certain other aspects, provided herein is a system for identifying one or more gene combinations associated with a trait, the system comprising one or more processors; and memory storing one or more programs, the one or more programs configured to be executed by the one or more processors, the one or more programs including instructions for: identifying one or more genetic variant combinations from a genetic data set based on one or more variant interaction scores, wherein each variant interaction score is representative of an association between all genetic variants of a single genetic variant combination of the one or more genetic variant combinations (a joint state of all genetic variants of a single genetic loci) and a shared trait, wherein the genetic data set comprises a plurality of inputs each having genetic information and at least one label indicative of the trait; identifying one or more genes associated with genetic variants of the one or more identified genetic variant combinations; selecting gene groupings from a library based on each gene grouping containing at least one of the identified one or more genes to form one or more gene grouping sets, wherein the library comprises groupings each representing an independent aspect of biology; determining an interaction-density score for each of the one or more gene grouping sets wherein the interaction-density score is based on normalization of a grouping interaction score determined from the variant interaction scores for the genetic variant combinations between gene groupings in the gene grouping set; and identifying the one or more gene combinations associated with the trait from at least one of the one or more gene grouping sets selected based on the determined interaction-density scores.

In some aspects, provided herein are methods for identifying a subpopulation of patients for drug response, the method comprising: identifying one or more genetic variant combinations from a genetic data set based on one or more variant interaction scores, wherein each variant interaction score is representative of an association between all genetic variants of a single genetic variant combination of the one or more genetic variant combinations (a joint state of all genetic variants of a single genetic loci) and a shared trait targeted by a drug, wherein the genetic data set comprises a plurality of inputs each having genetic information and at least one label indicative of the trait; identifying one or more genes associated with genetic variants of the one or more identified genetic variant combinations; selecting gene groupings from a library based on each gene grouping containing at least one of the identified one or more genes to form one or more gene grouping sets, wherein the library comprises groupings each representing an independent aspect of biology; determining an interaction-density score for each of the one or more gene grouping sets wherein the interaction-density score is based on normalization of a grouping interaction score determined from the variant interaction scores for the genetic variant combinations between gene groupings in the gene grouping set; and identifying the one or more gene combinations associated with the trait from at least one of the one or more gene grouping sets selected based on the determined interaction-density scores; wherein a gene in each of the one or more gene combinations is a target of the drug; identifying a subpopulation of patients with genetic variation in one or more genes in the identified one or more gene combinations.

### I. Definitions

Unless otherwise defined, all the technical terms used herein have the same meaning as commonly understood by one of ordinary skill in the art in the field to which this disclosure belongs.

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

The term “treating” or “treatment,” as used herein, is an approach for obtaining beneficial or desired results including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (e.g., partial or total) of the disease, decreasing the dose of one or more other medications required to treat the disease, delaying the progression of the disease, increasing the quality of life, and/or prolonging survival.

The term “individual” refers to a mammal and includes, but is not limited to, human, bovine, horse, feline, canine, mouse, rodent, or primate.

As used in this specification and the appended claims, the singular forms “a,” “an,” and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open-ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

The term “about” as used herein refers to the usual error range for the respective value readily known in this technical field. Reference to “about” a value or parameter herein includes (and describes) variations that are directed to that value or parameter per se. For example, description referring to “about X” includes description of “X.”

The section headings used herein are for organizational purposes only and are not to be construed as limiting the subject matter described.

### II. Methods

Provided herein, in certain aspects, are methods involving the identification of one or more gene combinations associated with a trait. In some embodiments, the methods disclosed herein use genetic data (such as human genetic data) to arrive at a list of target gene combinations (or derivatives thereof, such as expression products, e.g., RNA and polypeptides) that are associated with a trait. In some embodiments, the gene combinations (or derivatives thereof) are suitable for drug targeting and can provide a synergistic response to enable improved treatments including those based on subtherapeutic dosages and/or those having improved safety profiles (such as reduced toxicity) when compared to targeting a single target of the gene combination. In some embodiments, the gene combinations (or derivatives thereof) provide insights on factors underlying a specific trait, including a polygenic trait. Further, in certain other aspects, provided herein are methods for experimentally validating gene combinations (or derivatives thereof).

For purposes of brevity, the description herein focuses on information at the genetic level, e.g., genes and gene groupings. It is to be appreciated that the disclosure provided herein can be operated at different levels covering derivatives thereof, such as expression products, e.g., RNA and polypeptides. For example, in some embodiments, one or more proteins associated with genetic variants of one or more identified genetic variant combinations can be used in place of one or more proteins associated with the genetic variants of the one or more identified genetic variant combinations. Such approach to the description of the technology is not to be construed as limiting the teachings provided herein.

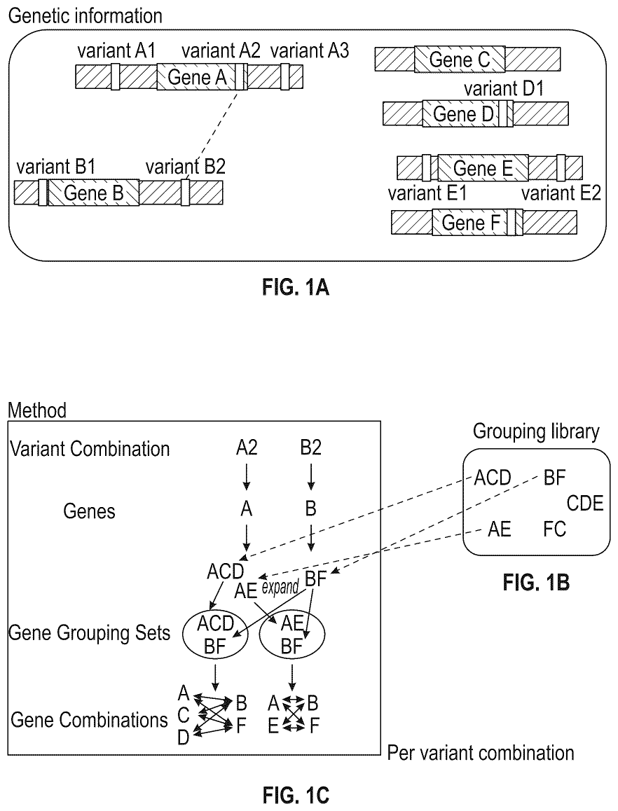

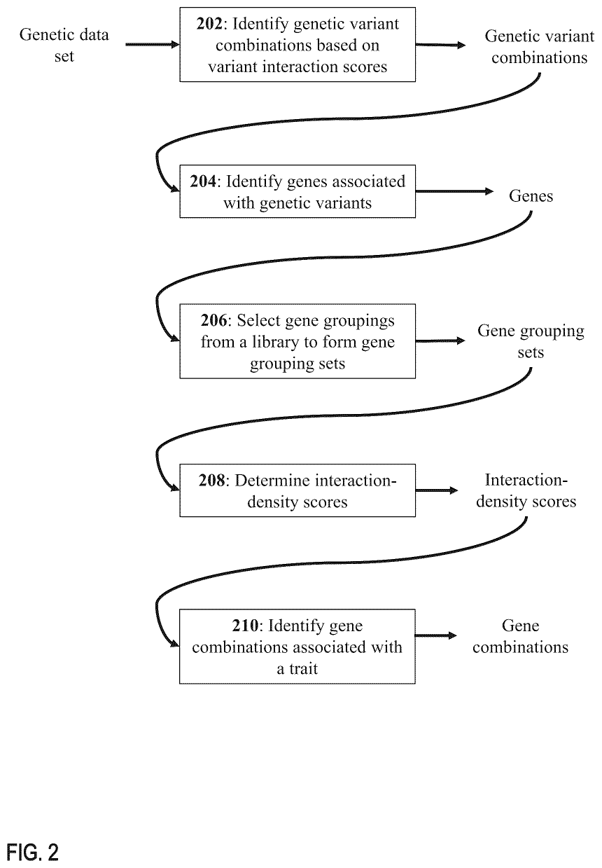

To assist with the understanding of the methods described herein, example workflows are illustrated in FIGS. 1 and 2. Such examples are not intended to limit the scope of the embodiments provided herein. For example, in some embodiments, provided in a method excluding one or more steps, or aspects thereof, illustrated in FIGS. 1 and 2. The illustration in FIG. 1 is based on one variant combination with a pair of variants, and it should be appreciated that the methods can be performed on several variant combinations and with variant combinations comprising more than two variants. As shown in FIG. 1, once a variant combination has been identified from genetic data based on a variant interaction score representative of an association between the two genetic variants and a shared trait, e.g., a human disease, the variants are assigned to genes. In some embodiments, a single variant may be associated with one gene (as shown in FIG. 1). In some embodiments, a single variant may be associated with two or more genes. Next, gene groupings are selected from a library based on each gene grouping containing the identified genes from the variants. The library contains groups of genes, such as a network or pathway. In some embodiments, a gene will appear in multiple groupings, and thus multiple gene groupings may be selected for each gene. Gene grouping sets are then compiled by permuting all of the possible combinations for the gene groups containing one gene and gene groups containing the other gene (in that regard the initial variant combination relationship guides these future relationships). Gene grouping sets (such as desired gene grouping sets based on an interaction-density score) are then permuted to identify gene combinations between the groupings. As noted herein, using the methods described herein a gene combination may or may not contain the gene that was originally mapped to the variant in the variant combination.

FIG. 2 provides an illustrative example workflow of method steps taught herein and indicates inputs and outputs to each step. As shown, the step that identifies genetic variant combinations by calculating variant interaction scores takes in the genetic data and outputs genetic variant combinations. The identifying genes step takes in the genetic variant combinations and associated output genes. The selecting gene grouping step takes in genes and outputs gene groupings from a library to form gene grouping sets. An interaction-density score can then be calculated as described herein, for example, by summing the variant interactions scores for all genetic variant combinations in all combinations of genes in the gene grouping set and normalizing the sum by a maximum value which can be determined empirically or by theory. Gene combinations are selected from the desired (e.g., those with high interaction-density scores as compared to a computed background distribution of interaction-density scores for genes that are not predicted to interact) gene groupings, wherein the gene combinations comprise one gene from each of the gene groupings.

At block 202, genetic variant combinations are identified based on interaction scores using a genetic data set as an input. In some embodiments, the genetic data set comprises information about gene variants across the genome for a plurality of inputs as described herein. The genetic data set may be processed using any of the cleaning methods described herein. In some embodiments, the genetic data set comprises a label indicative of the trait for which the method is being used to identify gene combinations. The label may be a categorical or continuous value indicative of a cellular, molecular, or clinical phenotype associated with the trait, as described herein. The label may be configured according to any of the labels indicative of the trait described herein.

The genetic variant combinations are identified at block 202 using any of the methods described herein for calculating a variant interaction score. In some embodiments, the variant interaction score represents a measurement of the synergy between genetic variants, wherein the synergy is an association between a trait and the joint state of the genetic variants of the combination as described herein. In some embodiments, calculating a variant interaction score for a combination of genetic variants comprises applying a decision rule as described herein. In some embodiments, the genetic variants identified at block 202 are genetic variants with significant variant interaction scores as described herein. In some embodiments, the genetic variant combination comprises 2 or more genetic variants as described herein.

At block 204, the genetic variant combinations identified at block 202 are used in identifying genes associated with the genetic variants. The methods at block 204 comprise mapping the genetic variants from the genetic variant combinations to one or more genes according to the methods described herein.

At block 206, the genes that were identified at block 204 are used in selecting gene groupings from a library to form gene grouping sets. The gene groupings may be selected by selecting gene groupings from a library of gene groupings that comprise at least one of the genes identified at block 204. In some embodiments, the library of gene groupings comprises any of the gene groupings described herein. In some embodiments, gene grouping sets are generated by combining gene groupings identified from the library. In some embodiments, generating the gene grouping sets comprise forming a set for all combinations of the gene groupings selected from the library as described herein and illustrated in FIG. 1B and FIG. 1C.

At block 208, the gene grouping sets selected at block 206 are used to determine interaction-density scores. In some embodiments, an interaction-density score is determined for one or more of the gene grouping sets. In some embodiments, the interaction-density score is generated from a grouping interaction score as described herein. In some embodiments, the methods comprise assessing the significance of the interaction density score for the gene grouping sets as described herein.

At block 210, the interaction density scores determined at block 208 are used to identify gene combinations associated with a trait. The gene combinations associated with the trait may comprise assessing significance of the interaction-density scores as described herein. In some embodiments, any of the filtering or prioritization methods described herein can be used to identify gene combinations associated with a trait at block 208.

Although not shown in FIG. 2, the gene combinations associated with a trait from block 210 may be experimentally validated. In some embodiments, experimental validation comprises genetic validation and/or pharmacogenetic validation as described herein. In some embodiments, the trait is a disease, or a phenotypic component of a disease, and the gene combinations may be druggable targets for the disease.

Although not shown in FIG. 2, the methods can be repeated for one or more traits and the outputs of the methods can be compared. In some embodiments, the methods can be used to identify overlapping gene combinations for two or more traits. In some embodiments, the methods can be used to identify traits related by gene regulation that had not previously been known to be related. In some embodiments, the methods can be used to identify gene combinations that are associated with multiple traits related to a single disease to prioritize gene combinations for validation and development as drug targets for the disease.

In some embodiments, the method provided herein is configured for identifying one or more genetic variant combinations from a genetic data set based on one or more variant interaction scores, wherein each variant interaction score is representative of an association between all genetic variants of a single genetic variant combination of the one or more genetic variant combinations (a joint state of all genetic variants of a single genetic loci) and a shared trait, wherein the genetic data set comprises a plurality of inputs each having genetic information and at least one label indicative of the trait; identifying one or more genes associated with genetic variants of the one or more identified genetic variant combinations; selecting gene groupings from a library based on each gene grouping containing at least one of the identified one or more genes to form one or more gene grouping sets, wherein the library comprises groupings each representing an independent aspect of biology; determining an interaction-density score for each of the one or more gene grouping sets wherein the interaction-density score is based on normalization of a grouping interaction score determined from the variant interaction scores for the genetic variant combinations between gene groupings in the gene grouping set; and identifying one or more gene combinations associated with the trait from at least one of the one or more gene grouping sets selected based on the determined interaction-density scores.

Further discussion of the methods and aspects thereof, taught herein, is included in the sections below. The modular discussion of such components does not limit the scope of the invention and one of ordinary skill in the art will readily appreciate how certain features from the sections below can be combined.

### A. Identifying One or More Genetic Variant Combinations From a Genetic Data Set Based on One or More Variant Interaction Scores

The methods provided herein, in certain aspects, comprise identifying one or more genetic variant combinations from a genetic data set based on one or more variant interaction scores. As detailed herein, each variant interaction score is representative of an association between all genetic variants of a single genetic variant combination of one or more genetic variant combinations (a joint state of all genetic variants of a single genetic loci) and a shared trait. In some aspects, the methods described herein comprise calculating one or more variant interaction scores.

A variant interaction score may measure the synergy between genetic variants (such as a pair of SNPs) and a trait. In some embodiments, synergy is an association between a trait and the joint state of the genetic variants of a genetic variant combination. In some embodiments, the state of the genetic variant may be a genotype or an allele. In some embodiments, the variant interaction score may be transformed or modified by a mathematical function. In some embodiments, the mathematical function may be a convolution with a shifted Heaviside function. In some embodiments, the mathematical function may be a threshold at a specific value, such as determined based on the context in which the method is being applied.

In some embodiments, the variant interaction score is based on detection of an interaction between two or more alleles as based on the presence of an effect arising from the joint state of the allele combination that is greater than would be expected by the sum of the individual effects. In some embodiments, the strength of the interaction can be modeled by way of a statistical model, or the like, or a causal model. For a statistical model, the strength may be expressed in terms of probabilities, odds ratios, and statistical significance of rejecting a null model. For a causal model, the strength may be expressed directly as the magnitudes of the coefficients of a parameter-adjusted and best-fit model. In some embodiments, the methods described herein involve an input of a collection of genetic variant data significance of the variant interaction score can be reached. Such data may include a volunteer cohort and/or data from a collection of cells, wherein the data comprise a label of a characteristic of the data source (such as the individual or cell, or a characteristic thereof).

In certain aspects, the variant interaction score is based on a logistical regression. In certain aspects, the variant interaction score is calculated using logistical regression. For example, a logistic regression may be used to score an interaction between two loci that impacts a binary phenotype. In some embodiments, the two alleles of each locus in an individual are translated to a single variable genotype state x1 and x2, for example under an additive model in which an allele takes on a value of 1 or 0 and therefore may have a state of 0, 1 or 2 (because there are two alleles). In some embodiments, a dominant model or a recessive model, in which the major or minor allele may play the role of either one and therefore can may have a state of 0 or 1. In some embodiments, the states of each locus go on to form the variables of a bivariate linear predictor, e.g., beta1*x1+beta2*x2+gamma*x1*x2+bias, the entirety of which is transformed by a logistic function. In some embodiments, the logarithm of the transformed value of the logistic function is then paired as a product with the observed phenotype in the individual. The negative sum of the product of the logistic function and the binary phenotype over all individuals may be the cross-entropy loss. Nonlinear methods may then be used to identify beta1, beta2, gamma and bias terms which yield both estimates of their values and the confidence intervals. In some embodiments, a gamma term with a non-zero value and a statistically significant test of being outside the interval consistent with the null hypothesis (zero interaction) would mean detection of an interaction effect that is significant over the sum of individual effects. In some embodiments, the interaction term is inferred rigorously by regressing on the residuals after conditioning out the individual effects and bias terms.

In certain aspects, the variant interaction score is based on a linear regression. In certain aspects, the variant interaction score is calculated using linear regression. For example, a linear regression may be used to score an interaction between two loci that impacts a continuous phenotype. In some embodiments, the two alleles of each locus in an individual are translated to a single variable genotype state x1 and x2, for example under an additive model in which an allele takes on a value of 1 or 0 and therefore may have a state of 0, 1 or 2 (because there are two alleles), a dominant model or a recessive model, in which the major or minor allele may play the role of either one and therefore can may have a state of 0 or 1. In some embodiments, the states of each locus go on to form the variables of a bivariate linear predictor, e.g., beta1*x1+beta2*x2+gamma*x1*x2+bias. The sum of squared differences between the continuous phenotype and the predicted phenotype may be the sum of squared errors loss. Linear methods may then be used to identify beta1, beta2, gamma and bias terms which yield both estimates of their values and the confidence intervals. In some embodiments, a gamma term with a non-zero value and a statistically significant test of being outside the interval consistent with the null hypothesis (zero interaction) would mean detection of an interaction effect that is significant over the sum of individual effects. In some embodiments, the interaction term is inferred rigorously by regressing on the residuals after conditioning out the individual effects and bias terms.

In other aspects, the variant interaction score is based on a hypergeometric test. In certain aspects, the variant interaction score is calculated using a hypergeometric test. For example, a hypergeometric test may be used to score an interaction between two loci when there is no detectable individual effect of either locus on a binary phenotype. In some embodiments, the two alleles of each locus in an individual are translated to a single variable state x1 and x2, for example under a dominant model or a recessive model in which the major or minor allele may play the role of either one and therefore can may have a state of 0 or 1. In some embodiments, there are therefore 4 possible genotypes comprising (0,0), (0,1), (1,0) and (1,1). Within any class (meaning all individuals of that class are grouped together), the count of diseased and healthy individuals may be given by n_D, and n_H. In some embodiments, it is then possible to determine, given the aggregate number of volunteers with disease and without disease (N_D, N_H), and the analogous number of volunteers with a given genotype state (n_D, n_H), using a hypergeometric test, whether there is an imbalance of case-controls in that joint allelic state.

In some aspects, the method comprises calculating one or more variant interaction scores. In some embodiments, calculating the variant interaction score for all of the one or more genetic variant combinations comprises applying different statistical tests for one or more genetic variant combinations that are tested. This aspect is based at least on the finding that different statistical tests are more powerful for detecting synergy between different genetic variant combinations and by applying a variety of statistical tests to assess a genetic variant combination, more synergistic genetic variant combinations can be identified. The methods described herein allow for increased flexibility of the method while maintaining statistical power to detect synergistic genetic variant combinations. The methods described herein are designed to incorporate genetic variant combinations identified using any or all of the statistical tests described herein, such as but not limited to a logistic regression, a linear regression, a hypergeometric test, a Fisher's Exact Test, or a Barnard's test.

In some aspects, the variant interaction score is based on a decision rule comprising more than one statistical test as described herein. In some aspects, the methods comprise calculating a variant interaction score using a decision rule. The decision rule may comprise applying a statistical test, and applying one or more additional statistical tests based on the results of the first statistical test. In some embodiments, the methods comprise a decision rule with one or more statistical tests described herein, such as but not limited to a logistic regression, a linear regression, a hypergeometric test, a Fisher's Exact Test, or a Barnard's test. In some aspects, the methods comprise generating a decision rule comprising one or more statistical tests.

In some embodiments, the variant interaction score is based on the association between two or more genetic variants and a disease as well as an association between each of two or more genetic variants and the trait. Testing the association between the combination of genetic variants and the trait as well as the association between each genetic variant individually allows for ensuring the effect of an individual genetic variant is not being identified falsely as a synergistic effect of the combination of the variants. In some embodiments, the association between the combination of two or more genetic variants and a trait as well as the association between each genetic variant individually and the trait can be tested using a single statistical test, such as any of the statistical tests described herein. In some embodiments, the association between the combination of two or more genetic variants and a trait, as well as the association between each genetic variant individually and the trait, can be tested using two statistical tests, such as any of the statistical tests described herein, such as but not limited to a logistic regression, a linear regression, a hypergeometric test, a Fisher's Exact Test, or a Barnard's test.

For purposes of illustration, in some embodiments, the genetic variant combination is a SNP-SNP pair, and the variant interaction score measures the synergy between the pair. In some embodiments, the synergy may be the association between a trait, such as a disease, and the joint state of the SNP-SNP pair. In some embodiments, the states of a SNP-SNP pair can be different, for example, the state for a SNP-SNP pair may be defined as “AA” or being in either “AA or AG,” where A refers to an adenine and G refers to a guanine respectively at each allele.

In some embodiments, the method comprises determining one or more mean and higher moments of a background distribution of interaction scores. In some embodiments the background distribution is a null distribution. In some embodiments, the method includes creating a background distribution of the mean interaction scores for all genetic variant combinations, wherein the distribution is ordered from least to most confident variant interaction scores or lowest to highest quality variant interaction scores. In some embodiments, confidence in a variant interaction score is the statistical significance representing how confident one can be that there is a true interaction between the genetic variants and the trait. In some embodiments the mean interaction scores for all genetic variant combinations are computed and the background distribution may be the lower 90% of the distribution. In some embodiments the mean interaction scores for all genetic variant combinations are computed and the background distribution may be the lower 99% of the distribution. In some embodiments the mean interaction scores for all genetic variant combinations are computed and the background distribution may be the lower 90%, 91%, 92%, 93%, 94%, 95%, 96%, 97%,98%, or 99% of the distribution. In some embodiments the mean interaction scores for all genetic variant combinations are computed and the background distribution may be calculated using statistical methods known in the art. In some embodiments, the statistical methods known in the art can identify a background distribution by removing outliers or identifying bimodal distributions.

In some embodiments, the method comprises creating a background distribution. In some embodiments, the method includes creating a background distribution of the variance in interaction scores for all genetic variant combinations, wherein the distribution is ordered from least to most confident variant interaction scores or lowest to highest quality variant interaction scores. In some embodiments, confidence in a variant interaction score is the statistical significance representing how confident one can be that there is a true interaction between the genetic variants and the trait. In some embodiments the background distribution is a null distribution. In some embodiments the variance in interaction scores for all genetic variant combinations are computed and the background distribution may be the lower 90% of the distribution. In some embodiments the variance in interaction scores for all genetic variant combinations are computed and the background distribution may be the lower 99% of the distribution. In some embodiments the variance in interaction scores for all genetic variant combinations are computed and the background distribution may be the lower 90%, 91%, 92%, 93%, 94%, 95%, 96%, 97%,98%, or 99% of the distribution. In some embodiments the variance in interaction scores for all genetic variant combinations are computed and the background distribution may be calculated using statistical methods known in the art. In some embodiments, the statistical methods known in the art can identify a background distribution by removing outliers or identifying bimodal distributions.

In some embodiments, the method includes creating a background distribution. In some embodiments, the background distribution represents the skewness of interaction scores for all genetic variant combinations, wherein the distribution is ordered from least to most confident variant interaction scores or lowest to highest quality variant interaction scores. In some embodiments, confidence in a variant interaction score is the statistical significance representing how confident one can be that there is a true interaction between the genetic variants and the trait. In some embodiments the background distribution is a null distribution. In some embodiments the background distribution is a null distribution. In some embodiments the skewness of interaction scores for all genetic variant combinations are computed and the background distribution may be the lower 90% of the distribution. In some embodiments the skewness in interaction scores for all genetic variant combinations are computed and the background distribution may be the lower 99% of the distribution. In some embodiments the skewness in interaction scores for all genetic variant combinations are computed and the background distribution may be the lower 90%, 91%, 92%, 93%, 94%, 95%, 96%, 97%,98%, or 99% of the distribution. In some embodiments the skewness in interaction scores for all genetic variant combinations are computed and the background distribution may be calculated using statistical methods known in the art. In some embodiments, the statistical methods known in the art can identify a background distribution by removing outliers or identifying bimodal distributions.

In some embodiments, the method includes creating a background distribution. In some embodiments, the background distribution represents the kurtosis of interaction scores for all genetic variant combinations, wherein the distribution is ordered from least to most confident variant interaction scores or lowest to highest quality variant interaction scores. In some embodiments, confidence in a variant interaction score is the statistical significance representing how confident one can be that there is a true interaction between the genetic variants and the trait. In some embodiments the background distribution is a null distribution. In some embodiments the background distribution is a null distribution. In some embodiments the kurtosis of interaction scores for all genetic variant combinations are computed and the background distribution may be the lower 90% of the distribution. In some embodiments the kurtosis in interaction scores for all genetic variant combinations are computed and the background distribution may be the lower 99% of the distribution. In some embodiments the kurtosis in interaction scores for all genetic variant combinations are computed and the background distribution may be the lower 90%, 91%, 92%, 93%, 94%, 95%, 96%, 97%,98%, or 99% of the distribution. In some embodiments the kurtosis in interaction scores for all genetic variant combinations are computed and the background distribution may be calculated using statistical methods known in the art. In some embodiments, the statistical methods known in the art can identify a background distribution by removing outliers or identifying bimodal distributions.

As discussed herein, in some embodiments, the methods are configured to identify one or more genetic variants. A diverse array of genetic variants are well known in the field and are encompassed by the description provided herein. In some embodiments, a genetic variant is a nucleic acid mutation. In some embodiments, the nucleic acid mutation may take the form of a single-nucleotide polymorphism (SNP), insertion, deletion, frameshift mutation, or copy number variation. A genetic variant may be associated with a variety of diseases or human conditions. Genetic variants can be determined using a variety of approaches (e.g., microarray, whole-genome sequencing, or whole-exome sequencing).

In some embodiments, the genetic variant combination is a combination of two or more genetic variants identified in a genome. In some embodiments, the genetic variant combination may comprise two or more SNPs. In some embodiments, the genetic variant combination may comprise two or more structural variants such as insertions or deletions. In some embodiments, the genetic variant combination may comprise two or more loci with copy number variation across individuals. In some embodiments, the genetic variant combination may comprise two or more genetic variants of different classes. In some embodiments, the genetic variant combination may be associated with a variety of diseases or human conditions. In some embodiments, the genetic variant combination may increase risk for a disease or have a protective effect thereby decreasing the risk of a disease.

In some embodiments, the one or more genetic variant combinations are between 1 and 1000 variant combinations. In some embodiments, the one or more genetic variant combinations may be about 1, 2, 3, 4, 5, 6, 7, 8, 9, 10, 20, 30, 40, 50, 60, 70, 80, 90, or 100 variant combinations. In some embodiments, the one or more genetic variant combinations may be about 100, 200, 300, 400, 500, 600, 700, 800, 900, or 1000 variant combinations.

In some embodiments, the one or more genetic variant combinations are between 1 and 100, 1 and 90, 1 and 80, 1 and 70, 1 and 60, 1 and 50, 1 and 40, 1 and 30, 1 and 20, or 1 and 10 variant combinations. In some embodiments, the one or more genetic variant combinations are between 1 and 900, 1 and 800, 1 and 700, 1 and 600, 1 and 500, 1 and 400, 1 and 300, 1 and 200, or 1 and 100 variant combinations

In some embodiments, the one or more genetic variant combinations each comprise between 2 and 50 variants. In some embodiments, the one or more genetic variant combinations each comprise 2, 3, 4, 5, 6, 7, 8, 9, 10, 20, 30, 40, 50, variants. In some embodiments, the one or more genetic variant combinations each comprise between 2 and 10, 2 and 20, 2 and 30, 2 and 40, 2 and 50, variants. In some embodiments, the one or more genetic variant combination comprises a SNP-SNP pair.

As described herein, an input to the disclosed method steps is a genetic data set. In some embodiments, the genetic data set comprises information about genetic variants across the genome for a plurality of inputs. In some embodiments, the genetic data set comprises information about genetic variants across the genome for one or more individuals. In some embodiments, the genetic data set comprises information about genetic variants across the genome for one or more single cells. In some embodiments, the genetic data set comprises the genomic coordinates of genetic variation and the ascertained allele at each coordinate for each of the individuals or single cell in the data set. In some embodiments, the genetic data set may comprise germline genetic variation. In some embodiments, the genetic data set may comprise somatic genetic variation. In some embodiments, the genetic data set may be obtained through genotype chips calls, exome arrays, whole genome sequencing, and/or single cell whole genome sequencing.

In some embodiments, the genetic data set is cleaned. Thus, in some embodiments, the method further comprises one or more steps of cleaning genetic data. Cleaning the genetic data set may include performing any one of several quality control steps. In some embodiments, principal component analysis is run on the data set to cluster similar inputs, and outlier inputs are removed. In some embodiments, principal component analysis is used to assess the totality of genetic markers in the input. In some embodiments, UMAP is used to assess the totality of genetic markers in the input. In some embodiments, outliers are defined as inputs that do not fall into a homogeneous population in the analysis assessing the totality of genetic makers. In some embodiments, the homogenous population may be a genetic ancestry, such as black, white, Japanese, or any other genetic ancestry. In some embodiments, the homogenous population may be cell type or cell state. In some embodiments, the cleaning can be completed using a GWAS tool such as PLINK.

In some embodiments, cleaning comprises removing inputs with missing data above about 2%. In some embodiments, cleaning comprises removing inputs with missing data above about 2%, about 1.5%, about 1.6%, about 1.7%, about 1.8%, about 1.9%, about 2.1%, about 2.2%, about 2.3%, about 2.4%, or about 2.5%. In some embodiments, cleaning comprises removing inputs with missing data above about 2%, about 3%, about 4%, about 5%, about 6%, about 7%, about 8%, about 9%, or about 10%. In some embodiments, cleaning comprises removing inputs with missing data between 2% and 100%, 2% and 90%, 2% and 80%, 2% and 70%, 2% and 60%, 2% and 50%, 2% and 40%, 2% and 30%, 2% and 20% or 2% and 10%. above about 2%.

In some embodiments, cleaning comprises removing genetic variants with missing data for above 2% threshold. In some embodiments, cleaning comprises removing genetic variants with missing data above about 2%, about 1.5%, about 1.6%, about 1.7%, about 1.8%, about 1.9%, about 2.1%, about 2.2%, about 2.3%, about 2.4%, or about 2.5%. In some embodiments, cleaning comprises removing genetic variants with missing data above about 2%, about 3%, about 4%, about 5%, about 6%, about 7%, about 8%, about 9%, or about 10%. In some embodiments, cleaning comprises removing genetic variants with missing data between 2% and 100%, 2% and 90%, 2% and 80%, 2% and 70%, 2% and 60%, 2% and 50%, 2% and 40%, 2% and 30%, 2% and 20% or 2% and 10%. above about 2%.

In some embodiments, cleaning comprises removing genetic variants that are represented in below about 5% frequency of inputs. In some embodiments, cleaning comprises removing genetic variants that are represented in below about 4.5%, 4.6%, 4.7%, 4.8%, 4.9%, 5%, 5.1%, 5.2%, 5.3%, 5.4%, or 5.5% frequency of inputs. In some embodiments, cleaning comprises removing genetic variants that are represented between 0% and 1%, 0% and 2%, 0% and 3%, 0% and 4%, or 0% and 5% frequency of inputs.

In some embodiments, cleaning comprises removing alleles represented below about 5% frequency in the inputs. In some embodiments, cleaning comprises removing alleles represented below about 4.5%, 4.6%, 4.7%, 4.8%, 4.9%, 5%, 5.1%, 5.2%, 5.3%, 5.4%, or 5.5% frequency in the inputs. In some embodiments, cleaning comprises removing alleles represented between 0% and 1%, 0% and 2%, 0% and 3%, 0% and 4%, or 0% and 5% frequency in the inputs.

In some embodiments, the genetic data sets are pruned as such removing some genetic variants. Pruning the genetic data may include identifying allelic variants such that no allelic pairs exhibit a linkage disequilibrium correlation exceeding 0.1 as computed by a control, genotyped cohort. Pruning the genetic data may include identifying allelic variants such that no allelic pairs exhibit a linkage disequilibrium correlation between 0 and 0.1 as computed by a control, genotyped cohort. Pruning the genetic data may include identifying allelic variants such that no allelic pairs exhibit a linkage disequilibrium correlation between 0 and 0.1, 0 and 0.01, 0 and 0.02, 0 and 0.03, 0 and 0.04, 0 and 0.05, 0 and 0.06, 0 and 0.07, 0 and 0.08, 0 and 0.09 computed by a control, genotyped cohort. The control, genotyped cohort may be from HapMap or 1000 Genomes. In some embodiments, PLINK methods can be used for LD pruning.

In some embodiments, the method described herein, the genetic sets are not pruned to remove some genetic variants. In some embodiments, the interaction testing is carried out using a sophisticated interaction model testing each variant against the trait, by removing variants to keep only those that show residual effects. In some embodiments, the sophisticated interaction model will account linked alleles so a priori pruning is not necessary. In some embodiments, the sophisticated interaction model performs fine mapping.

In some embodiments, a genetic data set comprises genetic data and at least one label indicative of a trait for a plurality of inputs. In some embodiments, the genetic data set comprises genetic data and at least one label indicative of a trait for a plurality of individuals. In some embodiments, the genetic data set comprises genetic data and at least one label indicative of a trait for a plurality of single cells from one or more individuals. In some embodiments, the genetic data set may comprise data from individuals with a disease and matched healthy individuals. In some embodiments, the genetic data set may comprise data from healthy individuals. In some embodiments, the genetic data set may comprise data from individual cells. In some embodiments, the genetic data set may comprise data from a CRISPR screen. In some embodiments, the genetic data set may comprise data from a CRISPR screen conducted in cell lines related to the trait. In some embodiments, the genetic data set may comprise data from a CRISPR screen conducted in a trait model system. In some embodiments, the genetic data set may comprise data from a CRISPR screen conducted in a disease model system.

In some embodiments, the genetic data set may comprise information from a published dataset. For example, the genetic data set may be downloaded from a biobank (e.g. UK Biobank, dbGAP). In some embodiments, the genetic data set may comprise information generated from an individual investigator. In some embodiments the genetic data set may comprise information generated by a consortium of investigators. In some embodiments, the genetic data set is assembled to comprise genotype chip calls, exome array, and/or other genetic markers accompanied by a label indicative of a trait.

In some embodiments, the label indicative of a trait is a label related to the trait in which the method is used to find associated gene combinations. For example, a label indicative of a trait may be a label describing a characteristic of a cell and/or individual from which the data are derived. In some embodiments, the label is binary, as in case or control status (e.g., disease versus non-disease). In some embodiments, the label is continuous, as in quantitative traits that can be at the molecular, cellular, or organismal level (e.g., height of an individual). In some embodiments, the label is the presence or absence of a disease. In some embodiments, the label is presence or absence of a common complex trait. In some embodiments, the label is used to indicate a disease and the disease may be metabolic, cardiovascular, immune, neuro-degenerative, other non-neuro-degenerative disease, or cancer. In some embodiments, the label is a metric (such as a measurement) clinically related to a trait. In some embodiments, the metric is collected by a healthcare provider in routine practice and/or in furtherance of a diagnosis.

In some embodiments, the label is a characteristic that may contribute to the trait. For example, the trait may be heart disease and the label indicative of the trait may be a binary classification if an input has heart disease. In some embodiments, the trait may be heart disease and the label indicative of the trait is a continuous measurement of BMI or cholesterol level.

### B. Identifying One or More Genes Associated With Genetic Variants

In certain aspects, the methods provided herein involve identifying one or more genes associated with genetic variants of one or more identified genetic variant combinations. In some embodiments, a genetic variant is associated with one gene. In some embodiments, the genetic variant is associated with two or more genes.

In some embodiments, the one or more genes are identified by mapping the genetic variants to genes, such as by using a reference genome. In some embodiments, genetic coordinates are used to map genetic variants to a gene. In some embodiments, the genetic coordinates are transcription start sites (TSS), transcription end sites (TES), annotated gene locations, or annotated transcription locations. In some embodiments, functional genomic data may be used to map genetic variants to genes. Functional genomics may include RNA-seq, ChiP-seq, ATAC-seq, DNA methylation, or chromatin looping. In some embodiments, the mapping is done with a machine learning model trained on data such as but not limited to Hi-C, eQTLs, pQTLs, and genomic coordinates. In some embodiments, the mapping methods output a confidence score for the mapping of the genetic variant to the gene. In some embodiments, a genetic variant may be mapped to more than one gene.

In some embodiments, the one or more genes are identified by mapping the genetic variants to genes, such as by using a reference genome. In some embodiments, the genetic coordinates are used to map genetic variants to a gene. In some embodiments, the genetic coordinates are in non-coding regions. In some embodiments, genetic variants in non-coding regions can be mapped to a gene. In some embodiments, genetic variants in non-coding regions can be mapped to a gene by identifying which gene the variant has a function effect one. A variant may be mapped to a gene because the variant impacts mRNA expression, mRNA splicing, translation speed, expressed protein function, expressed protein stability, or expressed protein level. A variant may be mapped to a gene because the variant impacts functional genomic data. Functional genomics may include RNA-seq, ChiP-seq, ATAC-seq, DNA methylation, or chromatin looping. In some embodiments, the one or more genes are the genetic variants of one or more identified genetic variant combination using published methods, such as PLINK, BOOST, CASSI, S-BEAM or SIPI. In some embodiments, the one or more genes are the genetic variants of one or more identified genetic variant combination using L2G. In some embodiments, the one or more genes are the genetic variants of one or more identified genetic variant combination using a machine learning algorithm. In some embodiments, the one or more genes are the genetic variants of one or more identified genetic variant combination using a machine learning algorithm trained on biological information derived from methods described herein.

### C. Selecting Gene Groupings From a Library

In certain aspects, the methods provided herein involve selecting gene groupings from a library based on each gene grouping containing at least one of the identified one or more genes to form one or more gene grouping sets. In certain aspects, provided herein are libraries comprising groupings each representing an independent aspect of biology. In some embodiments, as used herein, independent aspects of biology refer to two or more gene groupings having at least one non-overlapping gene. By including non-overlapping genes, gene groupings may represent independent pathways or gene networks that have been identified and validated molecularly.

In some embodiments, the gene groupings in a library represent independent aspects of biology. In certain aspects, an independent aspect of biology may be reflected by a gene grouping having at least one non-overlapping gene when compared directly to another gene grouping in the library. For example, a gene grouping comprising genes A, B, and C will be considered to have an independent aspect of biology when compared to a second gene grouping comprising genes A, B, and D. In some embodiments, the library comprises one or more gene groupings that are a gene network, or aspects thereof. In some embodiments, the library comprises one or more gene groupings that are a biological pathway, or an aspect thereof. In some embodiments, the library comprises one or more gene groupings based on co-expressed genes in a condition (such as genes known to be overexpressed in a disease state). In some embodiments, the library comprises one or more gene groupings based on genes related to one another based on a biological process (such as represented by a network or pathway). In some embodiments, the library comprises one or more gene grouping based on information from a database such as but not limited to Reactome, the Kyoto Encyclopedia of Genes and Genomes (KEGG), GO, Biocarta, WikiPathways, HPRD, STRING/IntAct, PANTHER, and ENCODE.

As noted herein, use of the term gene can be extended to derivatives thereof, such as expression products, e.g., RNA and polypeptides. Thus, a gene grouping may be based on a protein pathway or network, or an aspect thereof.

In some embodiments, the library comprises at least about 10 gene groupings, such as at least about any of 50, 100, 250, 500, 1000, 5,000, 10,000, 100,000, 200,000, 300,000, 400,00 or 500,000 gene groupings.

In some embodiments, the members of a gene group comprise a shared function, protein interactions, evolutionary relationships, common functional elements, association to a trait, and/or under-expression or overexpression in a condition (such as based on a disease tissue). In some embodiments, the library is assembled using functional data such as mRNA expression, protein expression, protein binding patterns, and/or 3D genomic interactions.

In certain aspects, the methods comprise forming gene grouping sets. In some embodiments, the gene grouping set is based on the expansion potential gene groups selected based on the presence of a gene associated with a genetic variant in a genetic variant combination (e.g., as shown in FIG. 1).

### D. Determining Interaction-Density Scores and Grouping Interaction Scores

In certain aspects, the methods provided herein involve determining an interaction-density score for one or more gene grouping sets. In certain aspects, the interaction-density score is based on normalization of a grouping interaction score determined from the variant interaction scores for the genetic variant combinations between gene groupings in the gene grouping set.

In some embodiments, the grouping interaction score is based on variant interaction scores for genetic variant combinations between gene groupings in a gene grouping set. In some embodiments, the grouping interaction score is a function that takes variant interaction scores for genetic variant combinations between gene groupings in a gene grouping set and outputs a computer or machine-learned output representing the connectivity of the gene groupings in the gene grouping sets. In some embodiments, the grouping interaction score is based on the genetic variant interaction scores all the genetic variant combinations in the gene groupings that make up the gene grouping sets. In some embodiments, a group interaction score is a score computed using the variant interaction score for all genetic variant combinations in all combinations of genes in a gene grouping set. In some embodiments, the grouping interaction score is the sum of the variant interaction score for all genetic variant combinations in all combinations of genes. In some embodiments, the grouping interaction score is the number of connections between the gene groupings in the gene grouping sets. In some embodiments, the grouping interaction score is related to the connectivity of the gene groupings in the gene grouping sets.

As described herein, interaction-density scores can be determined using information from the grouping interaction scores. For example, using grouping interaction scores for all possible combinations of the selected gene groupings in the gene grouping sets, each grouping interaction score is normalized to create an interaction-density score. In some embodiments, the normalization may be performed by dividing the grouping interaction score by the theoretical maximum number of genetic variant combinations in the gene groupings that make up the gene grouping sets. In some embodiments, the significance of an interaction-density score is calculated by comparing the interaction-density score to distribution of genetic variant interaction scores calculated above and a statistical test such as a chi-squared test. In some embodiments, the statistical significance of the gene grouping sets is calculated. In some embodiments, the statistical significance is assessed by creating a permuted background distribution of interaction-density scores. The permuted background distribution may be computed by shuffling the identities of all genetic variants and recomputing interaction-density scores. After repeating this step, N times, each interaction-density score is compared to the permuted distribution to obtain a p-value. In some embodiments, N is estimated as a number that allows the precision needed to discard false positives. In some embodiments, N is about the number of grouping combinations. In some embodiments, N is 1, 5, 10, 50, 100, 500, 1000, 5000, or 10,000.

In some embodiments, the gene grouping sets are ranked and filtered. In some embodiments, the filtering may include removing all gene grouping sets that do not score above 0.05 for the p-value. In some embodiments, the filtering may be removing gene groupings sets to achieve the desired false discovery rate (FDR), e.g. 5%. The gene grouping sets may be ranked according to their interaction-density score for processing and further steps disclosed herein. In some embodiments, the graph of groupings may be used to rank and filter the gene grouping sets.

In some embodiments, the size of the variant combination synergy space for a gene grouping is accounted for when calculating the statistical significance of the gene grouping set. In some embodiments, the size of the variant combination synergy space is a product of the number of genes in each gene grouping in the gene grouping set. In some embodiments, if there is one gene grouping in the gene grouping set, the size of the variant combination synergy space is the square of the number of genes in the gene group. In some embodiments, the size of the variant combination synergy space is a size range. In some embodiments, the size ranges are selected by evenly spacing the sizes of the variant combination synergy space for all gene grouping sets in logarithm space. For example, the first size range being of sizes 10-100, the second size range being sizes 100-1000, the third size range being of size 1000-10,000 etc. In some embodiments, accounting for the variant combination synergy comprises assigning a threshold determined from sampling a null genetic model based on the assigned size range. In some embodiments, the threshold is between about 0.1% and about 1%. In some embodiments, the null genetic model refers to a synthetically derived cohort of permuted case and control labels that retains allele states and is subjected to the same process of identifying synergistic gene groupings.

In some embodiments, interaction-density scores for groupings can be used to compare and prioritize groupings. In some embodiments, the interaction-density scores for groupings can be used to prepare a knowledge graph of groupings for prioritizing groupings for further analysis. The nodes would be the gene groupings and the edges would be the interaction-density score or a transformation thereof. In some embodiments, the relationship between the gene groupings can be accounted for and additional biologically relevant information can be considered. The iterative nature of the methods described herein can be understood here. In some embodiments, gene groupings with validated gene combinations for one or more diseases may be prioritized for further analysis. The methods may allow for identification of gene groupings that connect to more other gene groupings than expected and thus likely contain genes with strong global gene regulatory effects.

### E. Identifying Gene Combinations Associated With a Trait

In certain aspects, the methods provided herein involve identifying one or more gene combinations associated with a trait. In some embodiments, the identifying the one or more gene combinations associated with the trait is from at least one of the one or more identified gene grouping sets selected based on the determined interaction-density scores. In some embodiments, the method comprises comparing an interaction-density score with a permuted background distribution of interaction density scores to obtain a p-value and then selecting grouping sets that are above a desired p-value for downstream use identifying gene combinations associated with a trait. In some embodiments, the permuted background distribution is obtained by iteratively shuffling genetic variant identities and computing an interaction-density score.

In some embodiments, gene combinations are identified by enumerating all possible gene combinations from one or more gene grouping sets (such as those identified based on interaction-density scores, e.g., based on a ranking of interaction-density scores). In some embodiments, gene variant combinations having a variant interaction score are kept for downstream analysis and identification of a gene combination associated with a trait. As described herein, a gene combination can comprise any of 2 or more genes, including 2, 3, 4, 5, 6, 7, 8, 9, or 10 genes. In some embodiments, the gene combinations are gene-gene pairs, wherein each gene is from one of the two gene groupings in a gene grouping set. Such methodology translates to gene combinations comprising more than two genes. For example, in some embodiments, the gene combinations are one gene from each of three gene groupings in a gene grouping set, wherein each gene is from one of the three gene groupings in a gene grouping set.

In some embodiments, the one or more gene combinations are filtered and/or prioritized, such as according to the genetic variant interaction scores for all the genetic variant combinations in the gene combination. In some embodiments, the one or more gene combinations are filtered and/or prioritized by integrating expression data from human cells or tissues, expression data from cell lines, and/or druggability data. In some embodiments, the expression data are gene expression. In some embodiments, the expression data are RNA expression. In some embodiments, the expression data are protein expression.

In some embodiments, the methods described herein comprise ranking a list of genes in the gene groupings according to the sum of the genetic variant association scores for all genetic variant combinations in the gene grouping. In some embodiments, the ranked list is used to identify and/or prioritize gene combinations. In some embodiments, the gene combinations are filtered and/or prioritized based on their co-druggability. In some embodiments, co-druggability may be assessed using protein structure, protein binding pockets, and/or the level of disorder in the proteins.

In some embodiments, one or more genes in a gene combination will not be a gene originally mapped to an associated genetic variant combination. In some embodiments, one or more genes in a gene combination will not be a gene with identified genetic variants. In some embodiments, one or more genes in a gene combination will not be a gene with identified genetic variants associated with a trait.

### F. Cleaning and Filtering Techniques

In certain aspects of the description provided herein, the methods may comprise one or more cleaning and/or filtering techniques. The cleaning and/or filtering techniques can be applied at a number of different stages of the methods taught herein, some of which are discussed in other places in the disclosure. For example, in some embodiments, the method comprises performing a quality control technique on genetic data to form a genetic data set. For example, in some embodiments, the quality control technique comprises one or more of removing individuals based on distance away from a cluster (such as the largest cluster), removing individual missing data above a desired threshold, removing polymorphisms that are below a desired frequency threshold, and removing allelic variants that below a desired frequency threshold. In some embodiments, the method comprises pruning allelic variants such that no allelic pairs exhibit a linkage disequilibrium correlation exceeding a desired threshold as computed by a control, genotyped cohort.

In some embodiments, the methods comprise selecting a certain feature when it is above a background distribution. For example, in certain aspects, the methods comprise determining a background interaction-density score, such as derived from a statistical test (e.g., chi-squared) performed on computed mean and higher moments of calculated variant interaction scores. In some embodiments, a permuted background distribution is obtained by iteratively shuffling genetic variant identities and computing an interaction-density score. In some embodiments, the method comprises comparing an interaction-density score with a permuted background distribution of interaction density scores to obtain a p-value and then selecting grouping sets that are above a desired p-value for downstream use identifying gene combinations associated with a trait.

In some embodiments, the collection of gene combinations associated with a trait determined using a method described herein are further filtered and/or prioritized. For example, as described herein, such gene combinations may be filtered based on expression data and druggability. For example, if treating a disease in a specific tissue, one may remove a gene not expressed in said tissue. In some embodiments, one may remove a gene that is not druggable candidate, e.g., does not contain a binding pocket.

In some embodiments, the further filtering and/or prioritization of gene combinations comprises prioritizing gene combinations based on tractability of one or more gene in the gene combination for druggability. In some embodiments, information related to tractability of a gene for druggability comprise, genetic, biological, structural, and pharmacological data. In some embodiments, genetic data comprise associations with adverse events or intolerance to loss. In some embodiments, biological data comprise sequence information such as similarity between genes in the gene combination, and gene expression levels in one or more tissues. In some embodiments, structural data comprise x-ray crystallography data, NMR-determined sequence, and/or the presence of a ligand in the structure. In some embodiments, pharmacological data comprise compound interaction data. In some embodiments, a gene combination is prioritized if the tractability of each gene in a gene combination suggests each gene in the combination is druggable.

In some embodiments, the further filtering and/or prioritization of gene combinations comprises selecting genes combinations using synergy values (such as variant interaction scores) generated using the methods described herein. In some embodiments, the methods comprise ranking genes in a gene grouping by variant interaction scores for variants associated with the gene and selecting combinations with the top variant interaction scores. In some embodiments, the methods comprise ranking the combinations of genes from the gene groupings by the variant interaction scores for variant combinations between the genes in the combination and selecting the top combinations. In some embodiments, the methods comprise filtering and/or prioritizing genes combinations with higher than expected variant interactions scores. In some embodiments, machine learning models can be used to filter and/or prioritize gene combinations based on synergy values or validate synergistic gene combinations. In some embodiments, provided herein, machine learning methods can be used to predict features related to gene combinations that can be successfully validated experimentally, and such features can be used for filtering and prioritizing gene combinations.

### G. Integration of Results for Multiple Traits

In certain aspects, provided herein are methods that can be used to identify gene grouping sets or gene groupings associated with multiple traits. In some embodiments, the methods described herein can be performed for multiple traits and the resulting gene groupings can be compared. In some embodiments, the multiple traits relate to a single disease. Multiple traits related to a single disease may be different phenotypic aspects of a disease or different progression outcomes of a disease. Accordingly, the methods may be used to identify gene combinations that are relevant for regulation of different phenotypes associated with a single disease. The identified gene combinations can aid in the understanding of disease biology for complex diseases. The methods described herein can be used to identify gene combinations that when drugged improve symptoms more effectively for a subset of individuals with the disease. In some embodiments, gene combinations identified herein can be used for personalized medicine.

In some embodiments, the methods described herein can be performed for multiple traits relating to different diseases. Accordingly, the methods may be used to identify gene combinations that are relevant for multiple diseases and are druggable targets for treating multiple diseases. In some embodiments, the methods described herein can be performed sequentially for two or more traits. In some embodiments, the methods described herein can be performed in parallel for two or more traits.

In some embodiments, the relationship between two or more traits may be known. In some embodiments, the relationship between two or more traits may be a similar ontology. In some embodiments, the relationship between two or more traits may be unknown and the methods may be used to explore the relationship between two or more traits. In some embodiments, identifying gene combinations associated with multiple traits can inform common mechanisms for previously unconnected traits.

In some embodiments, the methods comprise generating a first set of gene groupings for a first trait; generating a second set of gene groupings for a second trait; determining an overlap between the first and second sets of gene groupings. The methods may comprise determining the overlap between the first and second set of gene groupings and generating gene combinations comprising genes in the first and second sets of gene groupings. An overlapping gene grouping and resulting gene combinations may be prioritized as related to both traits if a gene grouping from the first set shares more than a predetermined value of its genes with a gene grouping from the second set. In some embodiments, gene combinations for the prioritized gene groupings are experimentally validated. In some embodiments, the predetermined value is greater than 40%, greater than 50%, greater than 60%, greater than 70%, greater than 80%, or greater than 90%.

In some embodiments, gene combinations (e.g., prioritized gene combinations) identified for multiple traits may be driver gene combinations. In some embodiments, driver gene combinations may be prioritized for experimental validation. In some embodiments, driver gene combinations can be identified for multiple traits related to a single disease. The driver gene combinations may relate to an underlying gene regulatory mechanism for the disease and may provide a novel druggable target for the disease.

### H. Data Structures

In certain aspects, provided herein are data structures useful for performing the taught methods or aspects thereof.

For example, in some embodiments, the method comprises constructing and storing data in one or more knowledge graphs (KG). In some embodiments, the one or more KGs comprise a plurality of nodes and casual edges. The causal edges described herein are based on information regarding a known or predicted relationship between two nodes (in certain aspects, such a relationship is referred to herein as a causal relationship) and include information regarding the impact of an entity represented by a first node on another entity represented by a second node. As described herein, in some embodiments, the path may be characterized by an attribute thereof, such as length based on the number of nodes and/or the number of causal edges. In some embodiments, the length of a path is described by the number of causal edges. Generally, the KGs described herein may have paths of any length, and, in some embodiments, it may be desirable to limit the KG to paths having a maximum length. In some embodiments, the KG comprises paths of 20 or fewer causal edges, such as any of 19 or fewer causal edges, 18 or fewer causal edges, 17 or fewer causal edges, 16 or fewer causal edges, 15 or fewer causal edges, 14 or fewer causal edges, 13 or fewer causal edges, 12 or fewer causal edges, 11 or fewer causal edges, 10 or fewer causal edges, 9 or fewer causal edges, 8 or fewer causal edges, 7 or fewer causal edges, 6 or fewer causal edges, or 5 or fewer causal edges.

In some embodiments, the method described herein comprises constructing and storing data in a variant KG. In some embodiments, the variant KG comprises variant nodes, wherein each node is a genetic variant. In some embodiments, the causal edges are related to the relationship between variant nodes. In some embodiments, the causal edges are related to the variant combination scores. In some embodiments, the method described herein comprises constructing and storing data in a gene KG. In some embodiments, the gene grouping KG comprises gene nodes, where each node is a gene. In some embodiments, the edges are between the variants that show an interaction as measured by their variant interaction score. In some embodiments, the method described herein comprises constructing and storing data in a gene groupings KG. In some embodiments, gene groupings KG comprises gene grouping nodes, wherein each node is a gene grouping. In some embodiments, the causal edges are related to the relationship between the gene groupings. In some embodiments, the causal edges are interaction density scores.

In some embodiments, to reason over the data efficiently, a node-edge graph data structure of three tiers of increasing levels of complexity is used to hold the results of the analysis and enable navigation quickly. For example, at the first level, the variants are treated as a type of node, variant interactions are treated as another type of node, and edges connect variants and variant interactions, which enables representation of binary, ternary, and higher level interactions. At the second level, after variants are assigned to genes, genes are treated as a type of node and gene-gene interactions as another type of node and edges connect genes and interactions, which enables representation of binary, ternary, and higher level interactions. At the third level, genes are assigned to gene groupings in which gene groupings are treated as a type of node and gene grouping interactions as another type of node, and edges connect gene groupings to each other. At each level, feature data for variants, genes, and gene groupings are added to network representations to enable reasoning and prioritization of genes and gene groupings for downstream use, such as experimental validation.

In some embodiments, the method comprises storing a knowledge graph (KG) database on a device, such as a computer. In some embodiments, the KG database format comprises database nodes containing the information contained in a KG (such as described in more detail herein). The KG database format allows for information contained in a KG, such as stimulus nodes, biological entity nodes, condition nodes, and causal edges to be more quickly and efficiently accessed and analyzed on the device, such as the computer.

In some embodiments, the knowledge graphs described herein are created and stored using known software packages that are configured for creating and storing KG databases. For example, the knowledge graphs described herein can be created and stored using KG library software, such as Neo4j, Biological Expression Language (BEL), OrientDB, MongoDB, or ArangoDB. In some embodiments, the method comprises filtering the KG database using NetworkX, NetworkKit, igraph, enaR, ggnet2, or pajek.

### I. Traits

The methods provided herein are useful for studying any trait. In some embodiments, the trait is any discernable characteristic of a cell, tissue, and/or individual. In some embodiments, the trait is assessed at the molecular level. In some embodiments, the trait is a phenotype. The methods described herein are useful for studying a trait in an individual including, but not limited to, a human individual. In some embodiments, the trait is a human condition, such as a state of being and/or health (e.g., a disease state). In some embodiments, the trait is a metric (including a qualitative and/or quantitative metric) that is relevant to a human condition.

In some embodiments, the trait is a measurement clinically related to a human condition. In some embodiments, clinically related to a human condition may mean a measurement taken by a healthcare provider as a routine measurement or in furtherance of a diagnosis and/or treatment. In some embodiments, the trait is a binary trait (such as the presence or absence of a disease). In some embodiments, the trait is a continuous quantitative trait that can be measured at the molecular, cellular, or organismal level (such as body mass index and/or height).

In some embodiments, the trait is a disease. In some embodiments, the disease is a common complex trait. In some embodiments, a disease may be metabolic, cardiovascular, immune, neuro-degenerative, other degenerative, or cancer.

In some embodiments, the trait is a complication associated with a disease. In some embodiments, the trait is a subset of aspects associated with a disease. For example, the trait may be nephrovascular complications which are associated with a subset of individuals with type 2 diabetes. In some embodiments, the trait is a stage or form (e.g. advanced form) of disease. In some embodiments, the methods described herein can be used to identify gene combinations associated with disease progression of disease severity and/or a complication associated with a disease.

In some embodiments, the methods provided herein can be operated (sequentially or serially) on multiple traits. In some embodiments, the traits may be indicators of a common trait. For example, it is known in the art that blood pressure and BMI may be indicators of type two diabetes. In some embodiments, sequential use of the methods may help validate gene combinations for further testing as drug targets. In some embodiments, sequential use of the methods may broaden the number of gene combinations for further testing as drug targets.

### J. Experimental Validation

Provided herein, in certain aspects, are methods for experimentally validating one or more gene combinations associated with a trait. In some embodiments, the experimental validation is configured to assess a synergistic effect of a gene combination as compared to effects of its component parts. In some embodiments, the experimental validation is configured to assess the effect of a gene combination on a trait. In some embodiments, the experimental validation is configured to assess the effect of a gene combination on a phenotype measured at the cellular level that is associated with the trait. In some embodiments, the experimental validation comprises a cell-based assay, as described herein. In some embodiments, experimental validation comprises genetic validation as described herein. In some embodiments, experimental validation comprises pharmacogenetic validation as described herein. In some embodiments, experimental validation comprises genetic validation and pharmacogenetic validation. In some embodiments, experimental validation comprises pharmacological validation. Genetic validation is used to confirm the functional synergy predicted by genetic associations. Pharmacogenetic and pharmacological validation are used to show that predicted functional synergy for a gene combination can likely be achieved through drugging one or both genes or the polypeptides they encode. Pharmacogenetic validation can be used to show that a drug moiety itself is acting on the intended protein encoded by a gene in a gene combination and the drug moiety is not deriving its functional activity in the assay from other (ie cytotoxic/‘off-target’) mechanisms. Pharmacologic validation can be used to show that a combination (two or more) of drug moieties can achieve the predicted functional synergistic effect.

In some embodiments, the methods comprise experimentally validating the one or more gene combinations associated with a trait. In some embodiments, the methods comprise validation of the identified one or more gene combinations. In some embodiments, the identified one or more gene combinations comprise a gene combination comprising a first gene and a second gene. In some embodiments, the identified one or more gene combinations comprise a gene combination comprising a first gene, a second gene, and a third gene. In some embodiments, the identified one or more gene combinations comprise a gene combination comprising a first gene, a second gene, a third gene, and a fourth gene. It is appreciated that the gene combination may comprise more than four genes.

In some embodiments, the method comprises comparing measurable or observable metrics from an assay (e.g., cell-based assay) comprising modulating an activity and/or expression level of a gene combination and separately modulating an activity (e.g., by using a drug to modulate the biochemical activity of a protein encoded by a gene, or by using genetic methods to modulate the expression of a gene) and/or expression level of a subset of the gene combination (e.g., a single gene or each single gene of the gene combination). In some embodiments, the method comprises making one or more measurable or observable metrics following modulating an activity and/or expression level of a gene combination and/or one or more genes of a gene combination. In some embodiments, the method comprises: (a) modulating an activity and/or expression level of a gene combination and separately modulating an activity and/or expression level of a subset of the gene combination (e.g., a single gene or each single gene of the gene combination); (b) obtaining a measurable or observable metric from each modulated sample; and (c) comparing the measurable or observable metric to assess for the presence of synergy when modulating the gene combination as compared to a subset of the gene combination. In some embodiments, the observable metric is a phenotype as described herein associated with the trait.

In some embodiments, the assay is a cell-based assay. In some embodiments, the cell-based assay is selected from the group consisting of a cell viability assay, a cell growth assay, a cell proliferation assay, a growth inhibition assay, an ELISA assay, and a metabolic assay. In some embodiments, a cell-based assay comprises treatment of the cells in the cell-based assay to model one or more disease or trait conditions. In some embodiments, the cell-based assay comprises measuring a response in cells that have been subjected to a condition modeling one or more disease or trait conditions. In some embodiments, the cell-based assay comprises condition testing one or more conditions associated with a trait. For example, condition testing for type 2 diabetes may comprise testing of cellular response to high glucose environments. In some embodiments, the cell-based assay can be performed in cells, in tissue, or in one or more model organism.

In some embodiments, the methods comprise genetic validation. In some embodiments, provided herein is a genetic validation method comprising: (a) identifying one of more gene combinations associated with a trait according to any method provided herein; and (b) experimentally validating one or more of the identified gene combinations in a disease model system. In some embodiments, the identified gene combination is validated based on an observed phenotype consistent with a phenotype that may treat or prevent a human condition. In some embodiments, the phenotype comprises a characteristic based on one or more of cell growth inhibition, cytotoxicity, pro-or anti-apoptotic activity, inhibition or stimulation of a cellular stress response, such as induced by glucose or reactive oxygen species, modulation of glucose metabolism, modulation of insulin-dependent metabolism, production or inhibition of disease-related polypeptides, or cytokine production, immune cell co-culture toxicity assay changes, or mitochondrial activity changes. In some embodiments, the disease model system comprises a cell assay, an organoid assay, or an animal model. In some embodiments, the experimental validation comprises modulating an activity and/or expression level of an identified gene combination and comparing that to modulating an activity and/or expression level of one or more genes of the identified gene combination. In some embodiments, the experimental validation comprises a gene knockdown or knockout technique. In some embodiments, the gene knockdown or knockout technique is a siRNA technique, a CRISPR technique, zinc fingers, or Talens.

In some embodiments, experimentally validating comprises genetic validation. In some embodiments, experimentally validating comprises preforming a cell-based assay on a first cell sample subjected to a programmable genome or transcriptome modulator to modulate the expression of the first gene; a second cell sample subjected to a programmable genome or transcriptome modulator to modulate the expression of the second gene; and a third cell sample subjected to the programmable genome or transcriptome modulator to modulate the expression of the first gene and the programmable genome or transcriptome modulator to modulate the expression of the second gene, and determining if modulation of the expression of the first gene and the second gene results in a synergistic response observed in the cell-based assay. In some embodiments, the first cell sample and the second cell sample are further subjected to a non-targeting programmable genome or transcriptome modulator control. In some embodiments, the result of the cell-based assay comprises a phenotype related to the trait as described herein. In some embodiments, a synergistic response is an improved result in a cell-based assay as described herein when the combination of genes is modulated compared to the result of the cell-based assay when the genes are modulated individually. An improved response may be a phenotype associated with change in the trait or treatment of a condition associated with the trait.

In some embodiments, experimentally validating comprises preforming a functional assay on a first cell sample subjected to a programmable genome or transcriptome modulator to modulate the expression of the first gene; a second cell sample subjected to a programmable genome or transcriptome modulator to modulate the expression of the second gene; a third cell sample subjected to a programmable genome or transcriptome modulator to modulate the expression of the third gene; a fourth cell sample subjected to the programmable genome or transcriptome modulator to modulate the expression of the first gene, the programmable genome or transcriptome modulator to modulate the expression of the second gene, and the programmable genome or transcriptome modulator to modulate the expression of the third gene, and determining if modulation of the expression of the first gene, the second gene, and the third gene results in a synergistic response observed in the functional assay. In some embodiments, the first cell sample, the second cell sample and the third cell sample are further subjected to a non-targeting programmable genome or transcriptome modulator control. In some embodiments, the gene combination comprises more than three genes and the methods disclosed herein scales linearly to test the effect in a cell sample of a programmable genome or transcriptome modulator to modulate the expression of each gene in the combination. In some embodiments, the result of the cell-based assay comprises a phenotype related to the trait as described herein.

In some embodiments, the genetic experimental validation (e.g., cell-based assay) comprises a knockdown experiment. In some embodiments, the knockdown experiment is a dual knockdown experiment. For example, the experiment may include separately knocking down each gene in a gene combination and knocking down all of the genes in the gene combination. In some embodiments, the knockdown experiments can be performed with a programmable genome or transcriptome modulator as described herein. In some embodiments, the experiment is configured such that the read-outs can be measured to compare a knockdown of all genes of the first combination against at least a knockdown of a single gene of the first gene combination. In some embodiments, the knockdown is configured to perform the experiment on one of more of the gene combinations. In some embodiments, the knockdown experiment reduces an activity level and/or an expression level by at least about 20%, such as at least about any of 25%, 30%, 35%, 40%, 45%, 50%, 55%, 60%, 65%, 70%, 75%, 80%, 85%, 90% or 95%, as compared to a level prior to the knockdown. In some embodiments, the knockdown results are a difference from a non-targeting control (siNT). In some embodiments, the knockdown reduces activity level and/or an expression level by at least 20%, such as at least about any of 25%, 30%, 35%, 40%, 45%, 50%, 55%, 60%, 65%, 70%, 75%, 80%, 85%, 90% or 95%, as compared to the siNT.

In some embodiments, the genetic experimental validation (e.g., cell-based assay) comprises a knockout experiment. In some embodiments, the knockout experiments can be performed with a programmable genome or transcriptome modulator as described herein. In some embodiments, the knockout experiment is a dual knockout experiment. For example, the experiment may include separately knocking out each gene in a gene combination and knocking out all of the genes in the gene combination. In some embodiments, the experiment is configured such that the read-outs can be measured to compare a knockout of all genes of the first combination against at least a knockout of a single gene of the first gene combination. In some embodiments, the knockout is configured to perform the experiment on one of more of the gene combinations. In some embodiments, the knockout experiment substantially eliminates an activity level and/or expression level of a gene as compared to a level prior to the knockout.

Knockdown and knockout techniques are well known in the art for reducing an activity level and/or an expression level of a gene (or a derivative thereof). For purposes of this description, reduced encompasses when a gene activity level and/or expression level is substantially eliminated (e.g., only 2% of the original level of activity and/or expression exists following the knockdown or knockout). For example, in some embodiments, the activity level and/or expression level of a gene is reduced by mutating genomic DNA, reducing or inhibiting gene transcription, reducing mRNA species, reducing or inhibiting protein translation, modulating a post-translation modification that then reduces an activity level and/or expression level of a gene (or a derivative thereof). In some embodiments, the knockdown and knockout techniques include use of siRNA or other genetic engineering methods such as CRISPR, zinc fingers, or Talens. In some embodiments, knockdown and knockout techniques include use of a programmable genome or transcriptome modulator as described herein.

In some the programmable genome or transcriptome modulator for the first gene and the programmable genome or transcriptome modulator for the second gene are each selected from the group consisting of RNAi, a TALE-based modulation system, a zinc-finger-based modulation system, a meganuclease-based editing system, an epigenomic-based editing system, mRNA editing systems, and a CRISPR-based modulation system. In some embodiments, the RNAi is siRNA. In some embodiments, the programmable genome or transcriptome modulator comprise systems described herein for use in a knockdown experiment.

In some embodiments, experimentally validating comprises pharmacogenetic validation. In some embodiments, the experimental validating comprises performing a cell-based assay on: a first cell sample subjected to a drug moiety modulating the first gene, or an expression product thereof, and a non-target programmable genome or transcriptome modulator; and a second cell sample subjected to a drug moiety modulating the first gene and a programmable genome or transcriptome modulator targeting the second gene, and determining if there is an improved result in the cell-based assay from the second cell sample as compared to the first cell sample. In some embodiments, the result of the cell-based assay comprises a phenotype related to the trait as described herein.

In some embodiments, experimentally validating comprises pharmacological validation. In some embodiments, the experimental validating comprises performing a cell-based assay on: a first cell sample subjected to a drug moiety modulating the first gene, or an expression product thereof; a second cell sample subjected to a drug moiety modulating the second gene, or an expression product thereof; and a third cell sample subjected to a combination of the two said drug moieties modulating the each of the two genes, or expression products thereof, and determining if there is an improved result in the cell-based assay from the third cell sample as compared to the first two cell samples. In some embodiments, the result of the cell-based assay comprises a phenotype related to the trait as described herein.

In some embodiments, the one or more gene combinations comprise more than two genes. In some embodiments, the methods can scale linearly with the number of genes in the gene combination being validated. In some embodiments, the methods comprise successively subjecting cell samples to a drug moiety modulation for a gene in the combination and a non-target programmable genome or transcriptome modulator for the other genes in the gene combination and additional samples subjected to the drug moiety modulation for a gene in the combination and a programmable genome or transcriptome modulator for the other genes in the gene combination determining if there is an improved result in the cell-based assay when modulating each of the genes with the drug moiety and the on target programmable genome or transcriptome modulators for the other genes compared to modulating the gene with a drug moiety and off-target programmable genome or transcriptome modulators for the other genes.

For example, if an exemplary gene combination comprises gene A, gene B, gene C, a validation may be performed by: subjecting a first cell sample to a drug moiety modulation for gene A and a non-target programmable genome or transcriptome modulator for gene B and gene C, subjecting a second cell sample to a drug moiety modulation for gene A and a programmable genome or transcriptome modulator for gene B and gene C, subjecting a third cell sample to a drug moiety modulation for gene B and a non-target programmable genome or transcriptome modulator for gene A and gene C, subjecting a fourth cell sample to a drug moiety modulation for gene B and a programmable genome or transcriptome modulator for gene A and gene C, subjecting a fifth cell sample to a drug moiety modulation for gene C and a non-target programmable genome or transcriptome modulator for gene A and gene B, subjecting a sixth cell sample to a drug moiety modulation for gene C and a programmable genome or transcriptome modulator for gene A and gene B, and determining if there is an improved result in the cell-based assay for each of cell samples two, four, and six compared to cell samples one, three, and five.

In some embodiments, methods may comprise additional cell samples subjected to a drug moiety modulating two or more genes in a gene combination and a non-target programmable genome or transcriptome modulator and/or a programmable genome or transcriptome modulator targeting the remaining genes in the gene combination. In some embodiments, the drug moiety is a small molecule compound, peptide, protein, or antibody. In some embodiments, the drug moiety is an inhibitor. In some embodiments, the drug moiety is an activator.

In some embodiments, the experimental validation technique is based on a cellular assay or a cell-based assay. In some embodiments, the cellular assay comprises a cell that is relevant to a trait (such as the trait for which the association is based for identifying one or more gene combinations using the methods provided herein). In some embodiments, the cell lines may be selected because they have precedence in drug discovery for the trait. In some embodiments, the cells may be three cell lines all related to the trait.

The experimental validation techniques encompass many read-outs such as to obtain a measurable or observable metric (e.g., a metric associated with a characteristic). In some embodiments, the read-out is cell growth inhibition. In some embodiments, the read-out may be cell viability, apoptosis, cell death, or cytotoxicity. In some embodiments, the read-out may be cell growth inhibition, pro-or anti-apoptotic activity, inhibition or stimulation of cellular stress responses, such as by glucose or reactive oxygen species, modulation of glucose metabolism, modulation of insulin-dependent metabolism, production or inhibition of production of disease-related polypeptides, or cytokine production, immune cell co-culture toxicity assay changes, or mitochondrial activity changes.

In some embodiments, the read-out may be a change in a phenotype consistent with treating or preventing a human condition. In some embodiments, the phenotype may be cell viability, apoptosis, cell death, or cytotoxicity. In some embodiments, the read-out may be cell growth inhibition, pro-or anti-apoptotic activity, inhibition or stimulation of cellular stress responses, such as by glucose or reactive oxygen species, modulation of glucose metabolism, modulation of insulin-dependent metabolism, production or inhibition of production of disease-related polypeptides, or cytokine production, immune cell co-culture toxicity assay changes, or mitochondrial activity changes.

In some embodiments, the experimental validation comprises determining a synergy of a gene combination relative to a subset of the gene combination. In some embodiments, the Highest Single Agent (HSA) model is used, wherein the expected combination effect is the maximum effect of the best single gene effect, and the synergy is the difference between the combined effect and the HSA value. In some embodiments, the Bliss independence model is used, wherein the genes are expected to have independent effects and the expected effect of knocking down the group of genes is calculated based on the probability of independent events. Liu et al., Evaluation of drug combination effect using a Bliss Independence dose-response surface model. Stat Bipharm Res. 10(2), 112-122 (2018).

In some embodiments, the Bliss synergy is calculated by subtracting the bliss independence model value from the actual combined effect of the combinatorial knockout. In some embodiments, the Bliss synergy is calculated for gene combinations and the combinations are ranked. In some embodiments, the percentage of the experimental readout is calculated for gene combinations and the combinations are ranked. In some embodiments, gene combinations are validated if their synergy value is above 0. In some embodiments, gene combinations are validated if their synergy value is not negative. In some embodiments, gene combinations are validated if the combined impact on the experimental readout is greater than the experimental readout for the genes individually.

In some embodiments, the method provided herein comprises performing additional experiments for further validation of a gene combination. In some embodiments, qPCR is used to confirm the transcription knockdown or knockout level in a cell. In some embodiments, Western Blots may be used to confirm knockdown. In some embodiments, qPCR and Western Blots may be used to confirm knockdown. In some embodiments, a counter-screen is performed on mis-matched genes, wherein mis-matched genes are from groupings that were not predicted to interact according to methods disclosed herein. In some embodiments, the mis-matched genes are genes from mis-matched gene groupings (e.g. interactions predicted groupings A&B and C&D, genes within group A may be tested for interaction with genes in group C). In some embodiments, orthogonal phenotypic assays are used. In some embodiments, experiments are performed in additional cell line models that are not relevant to the trait.

### L. Additional Example Method Types

It is to be appreciated that the methods of identifying one or more gene combinations associated with a trait enable additional uses, which are also encompassed by the teachings provided herein. For example, in some embodiments, using the description taught herein, provided herein is a method of identifying one or more gene combinations (including derivatives thereof, such as expression products, e.g., RNA and polypeptides) associated with a trait, such as a human disease, that are co-druggable (either with a single agent or two or more agents, such as in a combination treatment). In some embodiments, the co-druggable targets (i.e., the one or more gene combinations or derivatives thereof) provide a synergistic benefit such as treatment of an individual having the trait. In some embodiments, the co-druggable targets (i.e., the one or more gene combinations or derivatives thereof) provide a treatment with a favorable safety profile.

In some embodiments, using the description taught herein, provided is a method of identifying one or more gene combinations (including derivatives thereof, such as expression products, e.g., RNA and polypeptides) associated with a trait, such as a human disease, such as to identify an underlying mechanism associated with said trait. For example, the methods provided herein can be used to identify one or more gene combinations (or derivatives thereof) associated with a phenotype of a polygenic human condition, such as a polygenic disease.

In some embodiments, using the description taught herein, provided is a method of identifying one or more gene combinations (including derivatives thereof, such as expression products, e.g., RNA and polypeptides) associated with a trait, such as a human disease, such as to provide one or more biomarkers of said trait. In some embodiments, the one or more biomarkers of said trait can be used for diagnostic or prognostic purposes. In some embodiments, the one or more biomarkers of said trait can be used for identifying a population of individuals suitable to receive a specified treatment. In some embodiments, the one or more biomarkers of said trait can be used for identifying a model (such as a cell or animal model) useful for studying the trait.

In some embodiments, using the description taught herein, provided is a method of identifying one or more gene combinations (including derivatives thereof, such as expression products, e.g., RNA and polypeptides) associated with one or more traits, such as one or more traits associated with a human disease, such as to provide one or more biomarkers of said one or more traits. In some embodiments, biomarkers identified using the methods here may be able to distinguish between two traits associated with the same disease. For example, the biomarkers identified may be associated with one trait of a disease but not another trait associated with the disease. Thus, in certain aspects, the biomarkers may be used to stratify patient populations within a disease genus, such as for clinical trials or treatments.

In some embodiments, the one or more biomarkers may interact with genes that are predicted to interact with a drug target. In some embodiments, a biomarker may be in the same gene grouping as a gene that is predicted to interact with a drug target. In some embodiments, the relationship between the biomarker and genes that are predicted to interact with a drug target can be used to stratify or select patients for treatment with the drug. In some embodiments, selected patients may have a better response rate or improved efficacy when treated with the drug.

In some embodiments, the one or more biomarkers can be used to stratify or select patients having a condition. In some embodiments, stratifying or selecting patients may include identifying patients with biomarker genotypes consistent with having a condition. In some embodiments, stratifying patients based on having a condition may help select the patient for the most effective care.

In some embodiments, the one or more biomarkers can be used to stratify or select patients for treatment with a treatment regimen. In some embodiments, stratifying or selecting patients may include identifying patients with biomarker genotypes predicted to impact response to a drug. In some embodiments, the biomarker genotype may be a loss of function mutation or a gain of function mutation. In some embodiments, the biomarker genotype may relate to better response rate or improved efficacy when treated with the drug.

In some embodiments, one of the genes in the gene combination may be a drug target. In some embodiments, the other genes in the gene combination may be used as markers to stratify or select patients. In some embodiments, patients with mutations in one of the genes in the gene combination may have a better response rate or improved efficacy when treated with the drug.

In some embodiments, provided is a method of identifying one or more biomarkers associated with a drug response, such as to identify an individual and/or a patient population who will react to a known drug. In some embodiments, the method further comprises obtaining, such as collecting clinical trial data for a drug, wherein the clinical trial data comprise a plurality of inputs each having genetic information and at least one label indicative of the inputs' response to a drug. In some embodiments, the label indicative of the inputs' response to a drug may be effectiveness in treating the trait. In some embodiments, the label indicative of the inputs' response to a drug may be a safety profile, e.g presence or absence of adverse events when taking the drug. In some embodiments, the method comprises identifying gene combinations associated with a trait. In some embodiments, the drug may be used to treat the trait. In some embodiments, the clinical trial data can be used to identify gene combinations using the methods described herein. In some embodiments, the genetic data used to identify gene combinations is a second genetic data set comprising a plurality of inputs each having genetic information and at least one label indicative of the trait the drug may be used to treat. In some embodiments, the method further comprises selecting the gene combinations that comprise a gene associated with the therapeutic activity of a drug, such as the gene encoding the protein, or the protein targeted by the drug. In some embodiments, the method further comprises selecting patients with genetic variants associated with genes in the selected gene combinations. In some embodiments, the method further comprises comparing drug efficacy in patients with genetic variants associated with genes in the selected gene combination to identify one or more biomarkers. In some embodiments, the one or more biomarkers is a gene. In some embodiments, the one or more biomarkers is a set of genes. In some embodiments, the one or more biomarkers may be associated with an increase in effectiveness of the drug. In some embodiments, the one or more biomarkers may be associated with a decrease in effectiveness of the drug. In some embodiments, the one or more biomarkers may be associated with a favorable safety profile, e.g., lower risk of adverse events as compared to all patients having the trait/disease. In some embodiments, the method may further comprise experimentally validating the biomarker. In some embodiments, experimentally validating the biomarker may include using the methods described herein to perform a knockdown of the biomarker gene. In some embodiments, the biomarker is experimentally validated if the response to the drug in the modified cell lines is different than the response to the drug in the non-modified cell lines or in a siNT control.

### III. Computer Implemented Methods and Systems

In certain aspects, provided herein are computer implemented methods for identifying one or more gene combinations associated with a trait (e.g. human disease). For example, the methods described in FIG. 2 and accompanying embodiments, can be implemented using one or more suitable computing devices capable of displaying a user interface to a user and recording and/or transmitting user inputs to a user interface. The computer implemented methods may be executed using the systems described herein.

In certain aspects, provided herein are systems configured for performing the methods, such as the computer implemented methods provided herein, or aspects thereof. In some embodiments, the systems may comprise one or more components, connected directly (such as by hardware) and/or indirectly (such as via wireless connection and/or data transfer). In some embodiments, the components of the system may communicate with one another using a network, such as the internet.

In some embodiments, the system comprises: one or more processors; and memory storing one or more programs, the one or more programs configured to be executed by one or more processors, the one or more programs including instructions for performing a method disclosed herein, or an aspect thereof. For example, in some embodiments, the instructions comprise information for performing a step of identifying one or more genetic variant combinations from a genetic data set based on one or more variant interaction scores according to the disclosure provided herein. In some embodiments, the instructions comprise information for determining a variant interaction score according to the disclosure provided herein. In some embodiments, the instructions comprise information for assembling a genetic data set according to the disclosure provided herein. In some embodiments, the instructions comprise information for identifying one or more genes associated with genetic variants of one or more genetic variant combinations according to the disclosure provided herein. In some embodiments, the instructions comprise information for selecting a gene grouping from a library according to the disclosure provided herein. In some embodiments, the instructions comprise information for determining an interaction-density score according to the disclosure provided herein. In some embodiments, the instructions comprise information for identifying one or more gene combinations based on an interaction-density score according to the disclosure provided herein.

In some embodiments, the system comprises one or more processors; and memory storing one or more programs, the one or more programs configured to be executed by the one or more processors, the one or more programs including instructions for: identifying one or more genetic variant combinations from a genetic data set based on one or more variant interaction scores, wherein each variant interaction score is representative of an association between all genetic variants of a single genetic variant combination of the one or more genetic variant combinations (a joint state of all genetic variants of a single genetic loci) and a shared trait, wherein the genetic data set comprises a plurality of inputs each having genetic information and at least one label indicative of the trait; identifying one or more genes associated with genetic variants of the one or more identified genetic variant combinations; selecting gene groupings from a library based on each gene grouping containing at least one of the identified one or more genes to form one or more gene grouping sets, wherein the library comprises groupings each representing an independent aspect of biology; determining an interaction-density score for each of the one or more gene grouping sets wherein the interaction-density score is based on normalization of a grouping interaction score determined from the variant interaction scores for the genetic variant combinations between gene groupings in the gene grouping set; and identifying the one or more gene combinations associated with the trait from at least one of the one or more gene grouping sets selected based on the determined interaction-density scores.

In some embodiments, the system comprises one or more machine learning models. In some embodiments, the instructions comprise running a machine learning model, such as trained with functional genetic data to identify the gene associated with a genetic variant.

In some embodiments, the one or more processors may comprise hardware processor such as a central processing unit (CPU), a graphical processing unit (GPU), a general-purpose processing unit, or other computing platform such as a cloud-based platform. The processor may be comprised of any of a variety of suitable integrated circuits, microprocessors, logic devices, field programmable gate array (FGPAs) and the like. In some embodiments, the reference to a processor may be to other types of integrated circuits and logic devices. The processor may have any suitable data operation capability.

In some embodiments, the storage can be any suitable device that provides storage, such as an electric, magnetic, or optical memory device including RAM, cache, hard drive, or removable disk. In some embodiments, storage can be in the form of an external computing cloud.

In some embodiments, the systems may comprise a user device. The user device may be a computing device configured to interface with various components of the system to control one or more tasks, cause one or more actions to be performed or effectuate other operations. In some embodiments, a user device may be a desktop computer, server, mobile computer, smart device, wearable device, cloud computing platform. In some embodiments, the user device may include one or more processors, memory, communications components, display components, audio capture/output devices, image capture components, or other components, or combinations thereof. The user device may include any type of wearable device, mobile terminal, fixed terminal, or other device.

### EXEMPLARY EMBODIMENTS

Embodiments disclosed herein may include:

Embodiment 1. A method for identifying one or more gene combinations associated with a trait,
the method comprising:
   identifying one or more genetic variant combinations from a genetic data set based on one or more variant interaction scores, wherein each variant interaction score is representative of an association between all genetic variants of a single genetic variant combination of the one or more genetic variant combinations and a shared trait,
      wherein the genetic data set comprises a plurality of inputs each having genetic information and at least one label indicative of the trait;
   identifying one or more genes associated with genetic variants of the one or more identified genetic variant combinations;
   selecting gene groupings from a library based on each gene grouping containing at least one of the identified one or more genes to form one or more gene grouping sets,
      wherein the library comprises groupings each representing an independent aspect of biology;
   determining an interaction-density score for each of the one or more gene grouping sets wherein the interaction-density score is based on normalization of a grouping interaction score determined from the variant interaction scores for the genetic variant combinations between gene groupings in the gene grouping set; and
   identifying the one or more gene combinations associated with the trait from at least one of the one or more gene grouping sets selected based on the determined interaction-density scores.

Embodiment 2. The method of embodiment 1, wherein the trait is the presence or absence of a human condition.

Embodiment 3. The method of embodiment 1, wherein the trait is a metric that is relevant to a human condition.

Embodiment 4. The method of embodiment 3, wherein the metric is assessed at the molecular, cellular, and/or organismal level.

Embodiment 5. The method of any one of embodiments 2-4, wherein the human condition is a disease.

Embodiment 6. The method of embodiment 5, wherein the human disease is a metabolic disease, a cardiovascular disease, an immune disease, a neuro-degenerative disease, a non-neuro-degenerative disease, or a cancer.

Embodiment 7. The method of any one of embodiments 1-6, wherein genetic variant combinations contain genetic variants wherein each is independently selected from the group consisting of SNP, structural variation, copy number variation, insertion, deletion, translocation, and inversion.

Embodiment 8. The method of any one of embodiments 1-7, wherein the genetic data set comprises an input assembled from genotype chip calls and/or exome arrays.

Embodiment 9. The method of any one of embodiments 1-8, wherein the genetic data set comprises an input from a published dataset.

Embodiment 10. The method of any one of embodiments 1-9, further comprising assembling the genetic data set.

Embodiment 11. The method of any one of embodiments 1-10, wherein the genetic data set, or a portion thereof, is cleaned.

Embodiment 12. The method of embodiments 11, wherein the genetic data set is cleaned by removing inputs outside a homogeneous population of inputs.

Embodiment 13. The method of embodiments 11 or 12, wherein the genetic data set is cleaned by removing inputs with missing genetic information above about 2%.

Embodiment 14. The method of any one of embodiments 11-13, wherein the genetic data set is cleaned by removing genetic information with missing information for above about 2% of inputs.

Embodiment 15. The method of any one of embodiments 11-14, wherein the genetic data set is cleaned by removing genetic information containing a variant below about 5% across the inputs.

Embodiment 16. The method of any one of embodiments 1-15, further comprising cleaning at least one of the plurality of inputs of the genetic data set.

Embodiment 17. The method of any one of embodiments 1-16, wherein the genetic variant combinations are cleaned by removing genetic variant combinations that have a linkage disequilibrium correlation exceeding about 0.1, wherein the linkage disequilibrium correlation is computed on a control genotyped cohort.

Embodiment 18. The method of any one of embodiments 1-17, further comprising cleaning the genetic variant combinations.

Embodiment 19. The method of any one of embodiments 1-18, wherein the variant interaction score is the synergy between the trait and a combined state of the genetic variant combinations.

Embodiment 20. The method of any of claims 1-19, wherein the variant interaction score relates to a logistic or linear regression that models the state of each genetic variant in the genetic variant combination as a as a bivariate linear predictor.

Embodiment 21. The method of any one of embodiments 1-20, further comprising transforming the variant interaction score.

Embodiment 22. The method of embodiment 21, wherein the transforming is done with a shifted Heaviside function or thresholding the values to exceed 0, whereby any negative values are converted to 0.

Embodiment 23. The method of any one of embodiments 1-22, wherein the identifying one or more genes associated with genetics variants of the identified plurality of genetic variant combinations is performed by inputting the genetic variants into a trained machine learning model configured to accept a genetic variant and output a gene.

Embodiment 24. The method of embodiment 23, wherein the machine learning model is trained using Hi-C, eQTL, pQTL, and genomic coordinates.

Embodiment 25. The method of any one of embodiment 1-24, wherein the library comprises gene groupings from one or more of Reactome, BioCarta, WikiPathways, Pathways Commons, HPRD, STRING/IntACT, PANTHER, ENCODE, or other groupings associated with a characteristic.

Embodiment 26. The method of any one of embodiments 1-25, wherein the library comprising groupings each representing an independent aspect of biology comprises one or more gene networks.

Embodiment 27. The method of any one of embodiments 1-26, wherein the library comprising groupings each representing an independent aspect of biology comprises gene groupings where each gene grouping has at least one non-overlapping gene when compared directly to another gene grouping in the library.

Embodiment 28. The method of any one of embodiments 1-27, further comprising assembling the library.

Embodiment 29. The method of any one of embodiments 1-28, wherein the grouping interaction score is a based on the variant interaction scores for the genetic variant combinations between the gene groupings in the gene grouping set.

Embodiment 30. The method of any one of embodiments 1-29, wherein the group interaction score is normalized by dividing the score by the theoretical maximum of the number of genetic variant combinations between gene groupings in the gene grouping set.

Embodiment 31. The method of any one of embodiments 1-30, wherein gene grouping sets are filtered out of the method if the interaction density score is below a computed background distribution.

Embodiment 32. The method of embodiments 31, wherein the computed background distribution is related to a permuted distribution of shuffled genetic variants combinations.

Embodiment 33. The method of any one of embodiments 1-32, wherein the gene combinations show gene expression differences in a trait relevant cell type or tissue.

Embodiment 34. The method of any one of embodiments 1-33, wherein the identified gene combinations are selected from gene grouping scores with an interaction-density score above the interaction-density scores in the computed background distribution.

Embodiment 35. The method of any one of embodiments 1-34, further comprising experimentally validating one or more of the identified gene combinations in a disease model system.

Embodiment 36. The method of embodiment 35, wherein an identified gene combination is validated based on an observed phenotype consistent with a phenotype that may treat or prevent a human condition.

Embodiment 37. The method of embodiment 36, wherein the phenotype comprises a characteristic based on one or more of cell growth inhibition, pro-or anti-apoptotic activity, inhibition or stimulation of a cellular stress response, modulation of glucose metabolism, modulation of insulin-dependent metabolism, or production or inhibition of disease-related polypeptides.

Embodiment 38. The method of any one of embodiments 35-37, wherein the disease model system comprises a cell assay, an organoid assay, or an animal model.

Embodiment 39. The method of any one of embodiments 35-38, wherein the experimental validation comprises modulating an activity and/or expression level of an identified gene combination and comparing that to modulating an activity and/or expression level of one or more genes of the identified gene combination.

Embodiment 40. The method of any one of embodiments 35-39, wherein the experimental validation comprises a gene knockdown or knockout technique.

Embodiment 41. The method of embodiment 40, wherein the gene knockdown or knockout technique is a siRNA technique or a CRISPR technique.

Embodiment 42. The method of any one of embodiments 1-41, further comprising selecting at least one of the identified gene combinations based on co-druggability.

Embodiment 43. A system for identifying one or more gene combinations associated with a trait, the system comprising:
one or more processors; and
memory storing one or more programs, the one or more programs configured to be executed by the one or more processors, the one or more programs including instructions for:
identifying one or more genetic variant combinations from a genetic data set based on one or more variant interaction scores, wherein each variant interaction score is representative of an association between all genetic variants of a single genetic variant combination of the one or more genetic variant combinations and a shared trait,
   wherein the genetic data set comprises a plurality of inputs each having genetic information and at least one label indicative of the trait;
identifying one or more genes associated with genetic variants of the one or more identified genetic variant combinations;
selecting gene groupings from a library based on each gene grouping containing at least one of the identified one or more genes to form one or more gene grouping sets,
   wherein the library comprises groupings each representing an independent aspect of biology;
determining an interaction-density score for each of the one or more gene grouping sets wherein the interaction-density score is based on normalization of a grouping interaction score determined from the variant interaction scores for the genetic variant combinations between gene groupings in the gene grouping set; and
identifying the one or more gene combinations associated with the trait from at least one of the one or more gene grouping sets selected based on the determined interaction-density scores.

Embodiment 44. A method for identifying a subpopulation of patients for drug response, the method comprising:
identifying one or more genetic variant combinations from a genetic data set based on one or more variant interaction scores, wherein each variant interaction score is representative of an association between all genetic variants of a single genetic variant combination of the one or more genetic variant combinations and a shared trait targeted by a drug,
   wherein the genetic data set comprises a plurality of inputs each having genetic information and at least one label indicative of the trait;
identifying one or more genes associated with genetic variants of the one or more identified genetic variant combinations;
selecting gene groupings from a library based on each gene grouping containing at least one of the identified one or more genes to form one or more gene grouping sets,
   wherein the library comprises groupings each representing an independent aspect of biology;
determining an interaction-density score for each of the one or more gene grouping sets wherein the interaction-density score is based on normalization of a grouping interaction score determined from the variant interaction scores for the genetic variant combinations between gene groupings in the gene grouping set; and
identifying the one or more gene combinations associated with the trait from at least one of the one or more gene grouping sets selected based on the determined interaction-density scores;
   wherein a gene in each of the one or more gene combinations is a target of the drug;
identifying a subpopulation of patients with genetic variation in one or more genes in the identified one or more gene combinations.

### EXAMPLES

### Example 1

This example demonstrates a method for identifying one or more gene combinations associated with a human trait, namely, a pancreatic cancer. Specifically, using the methods taught herein, genetic variant information was used to generate a list of polypeptide pairs (as derived from the one or more gene combinations) associated with pancreatic ductal adenocarcinoma (PDAC). The methodology demonstrated herein was designed to identify polypeptide pairs not otherwise known to be associated with PDAC, such as would be typically identified from conventional polygenic studies known at the time of filing this application.

A genetic data set was compiled from two independent data sets downloaded from dbGAP and UKBB. The genetic data comprised genotype data and individual labels indicating case-control status of pancreatic ductal adenocarcinoma (PDAC). The data contained SNP level information for individuals with PDAC and individuals not having PDAC (healthy control). The full data set contained 1919 cases and 2016 controls after quality control.

The genetic data set was cleaned and filtered for quality control (QC) purposes. Individuals with greater than 2% missing SNPs and SNPs with greater than 2% missing individuals were removed. SNPs with minor allele frequency below 5% were removed. Using a principal component analysis, individuals that did not cluster based on homogeneous populations in the first two principal components were removed. Using linkage disequilibrium data from HapMap and the 1000 Genomes Project, SNPs with linkage disequilibrium of r²>0.1 were removed to retain only the most desired SNPs for purposes of this study.

Next, a single pass SNP association GWAS was carried out independently on the genetic data originally obtained from dbGAP and from UKBB. The resulting associations were compared to ensure the data from the two independent studies replicated known loci.

Genetic variant combinations were identified using variant interaction scores. Variant interaction scores were derived using a binary interaction analysis performed for all SNP-SNP pairs. The interaction analysis was performed by detecting an interaction between two or more alleles that is defined as the presence of an effect arising from the joint state of the allele combination that is greater than would be expected by the sum of individual effects. The strength of the interaction was assessed using a hypergeometric test. The two alleles of each locus in an individual were translated to a single variable state x1 and x2, for example under a dominant model or a recessive model in which the major or minor allele may play the role of either one and therefore can may have a state of 0 or 1. There were therefore 4 possible genotypes comprising (0,0), (0,1), (1,0) and (1,1). Within any class (meaning all individuals of that class are grouped together), the count of disease and healthy is given by n_D, and n_H. Then a hypergeometric test was used to test if there was an imbalance of case-controls in the allelic state.

The one or more genes associated with the genetic variant's combinations were identified by identifying the genes associated with individual SNPs. Individual SNPs were mapped to genes using 4 data-driven approaches. First, one or more of the following local features of the SNP were used to prioritize target genes of interest: distance to transcriptional start site, presence of eQTLs and pQTLs in cell types, tissues, Hi-C association of the SNP region to regions proximal to the gene body, and global features of the SNP and target genes: concordance of biology of the gene as reported in literature or derived from data to the phenotype. Second, one or more of the local features were combined using a machine-learning method known as L2G to prioritize target genes of interest. Third, global features using a machine-learning method known as PoPs were used to prioritize genes of interest. Fourth, the outputs of the local and global methods were combined and trained as a meta-predictor to prioritize genes of interest.

Gene networks were combined to create a library of gene groupings resulting in ˜511 networks. The individual gene groupings represented independent aspects of biology. SNPs not associated with genes in a pre-defined library of gene groupings were removed.

Gene grouping pairs were identified by selecting gene groupings containing genes with significant variant interaction scores from the pre-defined library of gene groupings. The analysis resulted in ˜262,000 interacting networks.

Interaction density scores were determined for each of the gene grouping pairs. The interaction density score was the density of binary edges. The density of binary edges was computed for all gene grouping pairs and the interactions were filtered to those exhibiting a statistically significant enrichment of SNP-SNP edges compared to the expected density of 5%. Analysis results were organized into the node-edge graph network structure.

Gene grouping pairs were further filtered to remove pairs in which the genes appeared in both gene groupings originally.

The top 48 prioritized network pairs were selected by gene grouping node-edge graph and ensuring that a subset of gene grouping pairs were spread over the network, and a subset were concentrated near highly connected nodes, which enables testing of both diverse biology and also unusually well-represented biology by virtue of their high connectivity.

Genes from the gene grouping pairs were selected for pairwise experimental validation by choosing genes with expression in normal pancreas tissue, high and specific expression in tumorous pancreas tissue, genes that are small molecule druggable due to the presence of binding pockets and structural availability, genes with minimal genetic safety signals defined by Mendelian and non-Mendelian genetic association to phenotypes that are not related to pancreatic cancer, genes with literature support in connection to pancreatic cancer.

### Example 2

This example demonstrates two studies for the experimental validation of certain gene pairs identified from Example 1.

In the second study, a cell assay was optimized by selecting cell models with relevance in pancreatic cancer. BxPC-3, PANC-1, and MIA-PaCa2 cancer lines were selected to test because they have distinct genetic backgrounds and precedence in drug development. A cell proliferation assay and transfection conditions were optimized. The assay was performed by seeding 2.5×10³ cells in 96 well plates. 100 nM total siRNAs were reverse transfected with RNAiMAX™ into duplicated wells of the plate and with duplicate plates according to the following. 234 prioritized genes across top ranked networks were tested in the primary screen by knocking down those genes with siRNA. Wells for assessing individual genes received 50 nM non-targeted siRNA and 50 nM experimental gene targeted siRNA. Wells for assessing gene pairs received 50 nM experimental gene 1 and 50 nM experimental gene 2 siRNAs. At 96 hours, cells were lysed and assessed for ATP as a surrogate for cell count by CellTiter-Glo™. Cell growth was calculated by comparing signal at endpoint (96 hours) to signal at initiation (0 hours).

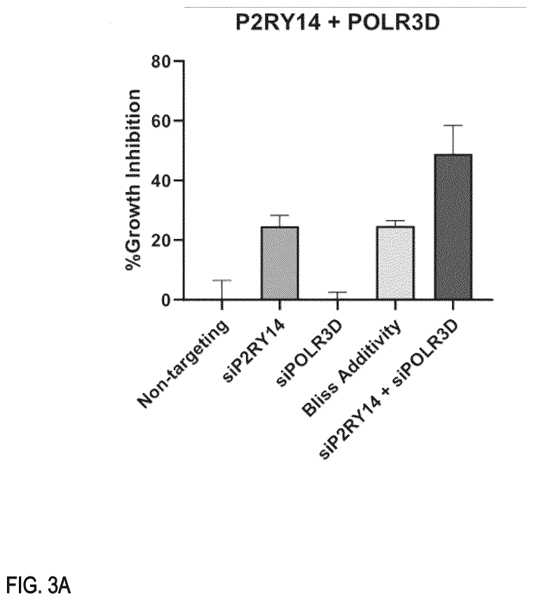

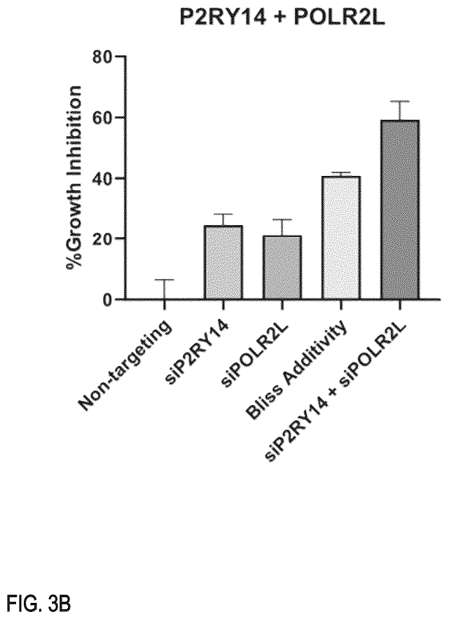

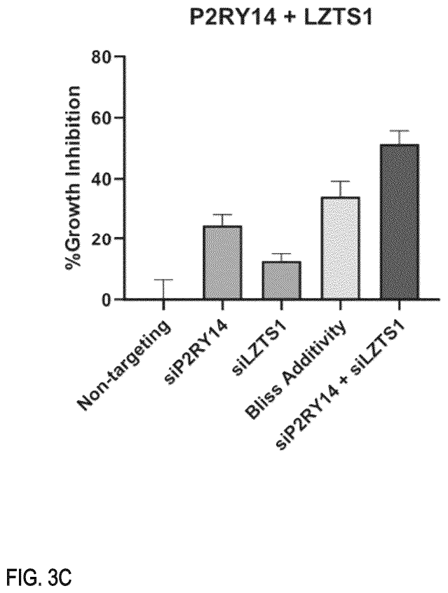

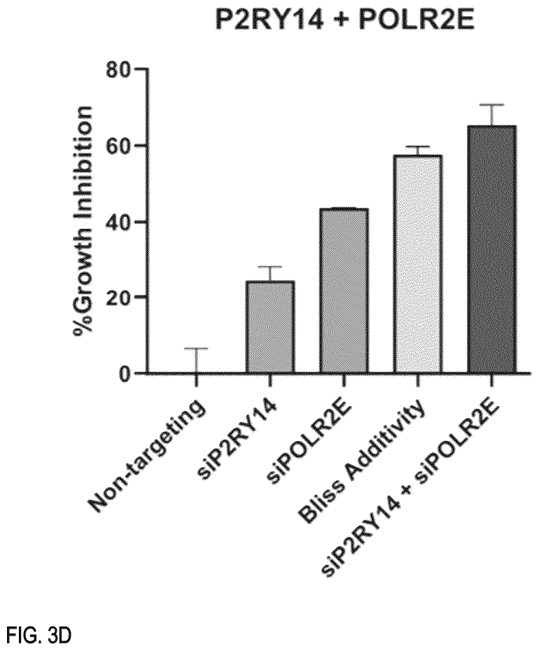

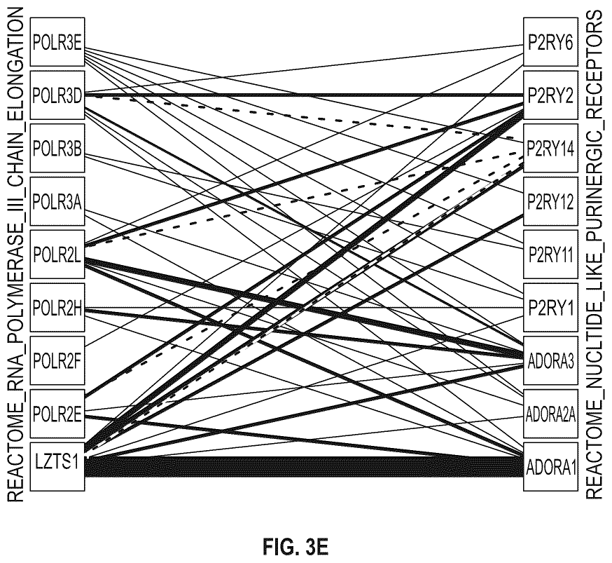

Single gene and dual gene knockdowns were compared. All samples were normalized to growth of the non-targeting control using the NCI growth calculation, as described by the National Cancer Institute's published Screening Methodology (accessed Dec. 28, 2023, https://dtp.cancer.gov/discovery_development/nci-60/methodology.html). Data were collected as inverse-growth inhibition such as 100% growth inhibition was equivalent to no growth in the non-targeting control from day 0 to the endpoints. Cytotoxicity was indicated by growth inhibition that exceeded 100%. In comparing non-target controls, background was considered above 20% growth inhibition. Co-druggable target hits were defined as gene pairs for which individual gene knockdowns showed little effect yet dual gene knockdowns showed an effect. Synergy was calculated for each pair by calculating the difference of the observed dual gene effect from the addition of two independent single gene effects, S_{Bliss}=E_{A,B}−100(1−(1−E_{A}100)(1−EB100)) (Liu et al, Stat Biopharm Res 2018). Specific data of top synergistic hits (P2RY14+POLER3D, P2RY14+POLR2L, P2RY14+LZTS1, and P2RY14+POLR2E) are provided in FIG. 3A-FIG. 3D. Synergy was calculated by the difference of the observed dual gene effect (darkest bar) from the addition of two independent single gene effects The highly ranked network pair, FIG. 3E, predicted the interaction of REACTOME_RNA_POLYMERASE_III_CHAIN_ELONGATION and REACTOME_NUCLEOTIDE_LIKE_PURINERGIC_RECEPTORS. SNP-SNP connections from the methods described in example 1 are shown with thicker edges denoting more significant interactions. The four highly synergistic gene pairs from panel B are shown by dashed lines.

### Example 3

This example demonstrates an exemplary bi-directional, drug moiety-based experimental pharmacogenetic validation suitable for assessing one or more gene combinations associated with a trait identified using the teachings herein.

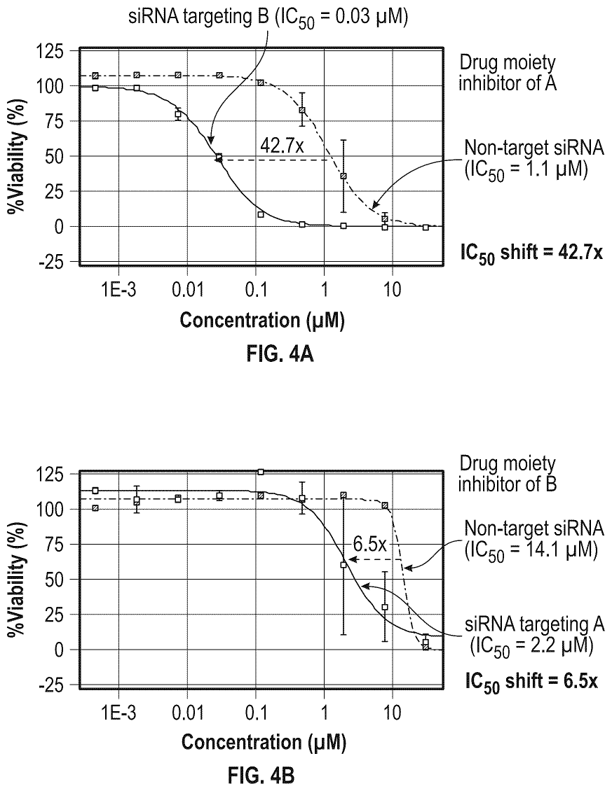

A gene combination was identified as A and B. Commercially available drug moieties that act as inhibitors of A and B, respectively, were obtained. Non-target siRNA (negative control), siRNA targeting A, and siRNA targeting B were obtained. Aliquots of cell samples from a disease model were cultured and then treated such as to evaluate (a) drug moiety inhibitor of A at varying concentrations plus non-target siRNA at a single concentration; (b) drug moiety inhibitor of A at varying concentrations plus siRNA targeting B at a single concentration; (c) drug moiety inhibitor of B at varying concentrations plus non-target siRNA at a single concentration; (d) drug moiety inhibitor of B at varying concentrations plus siRNA targeting A at a single concentration. After the treatment, a cell viability assay was performed. Data from use of the drug moiety inhibitor of A and the drug moiety inhibitor of B were plotted as shown in FIG. 4A and FIG. 4B, respectively. In both plots, the combination of the drug inhibitor for one member of the gene combination and the siRNA for the other resulted in a shift toward lower concentrations/more potent for the cognate siRNA as compared to the negative control siRNA. A shift toward improved potency is consistent with the notion that the drug moiety inhibitor of A phenocopies the siRNA. The shift is also consistent with the notion that the drug moiety inhibitor of A, because of its action, becomes more potent only in the presence of the properly paired siRNA predicted to inhibit the synergy. The results indicated that the compound itself is acting on the intended protein of the gene in the pair and the compound is not deriving its functional activity in the assay from other (ie cytotoxic/‘off-target’) mechanisms.

While evaluating the impact of using a drug moiety is necessary for only one of the targets, the analogous experiment with drug moiety inhibitor of B and an siRNA that knocks down gene A (i.e., bi-directional, drug moiety-based experimental validation) was also performed. As shown in FIG. 4B, there was a significant shift toward improved potency. This result showed that in addition to observing functional synergy when both genes are partially ablated with siRNA, that binding to the protein(s) achieves the same ends. The positive bidirectional effects showed the compound's mechanism of action works through the synergistic gene pair rather than through off-target effects. As such, the drug moiety-based experimental validation shows the functional synergy of the gene combination predict by genetics to be pharmacogenetically validated in a bi-directional manner.

### Example 4

This example demonstrates a method for identifying one or more gene combinations associated with micorvascular complications in type 2 diabetes (T2D). Microvascular complications include nephropathy, retinopathy, and neuropathy. Using the methods taught herein, genetic variant information was used to generate a list of gene combinations associated with microvascular complications in T2D, defined by the concurrent presence of renal and retinal complications.-Using the methods taught herein, genetic variant information was used to generate a list of gene combinations associated with renal and retinal complications in T2D. The overlap of the nephropathy and retinopathy network pairs was identified as an additional set of microvascular network pairs.

A genetic association data set was obtained from UK Biobank comprising genotype data assayed with a SNP array ICD10 diagnostic codes, and biomarkers indicating case-control status for a group of individuals with T2D. Nephropathy cases were defined as individuals with T2D who had developed nephrovascular complications and controls were defined as individuals with T2D who had not developed nephrovascular complications. Specifically, cases were defined as individuals with a urinary albumin-to-creatine ratio (uACR) of greater than 2.5 mg/mmol in women or greater than 3.5 mg/mmol in men and/or an estimated glomerular filtration rate (cGFR) of less than 60 mL/min/1.73 m². Controls were defined as individuals with uACR of less than or equal to 2.5 mg/mmol in women or less than or equal to 3.5 ng/mmol in men and eGFR of greater than 60 mL/min/1.73 m². To ensure the data from the study could be used to identify factors related to complications, individuals defined as cases and controls both were required to have an HbA1c level above 8%, consistent with a clinical diagnosis of T2D, irrespective of the individual's formal diagnosis status. Retinopathy cases were defined as individuals with T2D who had developed retinal complications and controls were defined as individuals with T2D who had not developed retinal complications. Specifically, cases were defined as individuals with the ICD10 code E11.3 (diabetic individuals with ophthalmic complications). To ensure the data from the study could be used to identify factors related to complications, individuals defined as cases and controls both were required to have an HbA1c level above 7.5%, consistent with a clinical diagnosis of T2D, irrespective of the individual's formal diagnosis status. The HbA1c level allowed was reduced compared to the nephropathy complication individuals to increase sample size. Microvascular complication cases were defined as individuals with T2D who had developed renal and retinal complications and controls were defined as individuals with T2D who had not developed renal or retinal complications. Individuals defined as cases and controls both were required to have an HbA1c level above 6.5%. The lower HbA1e level was selected to achieve an adequate sample size.

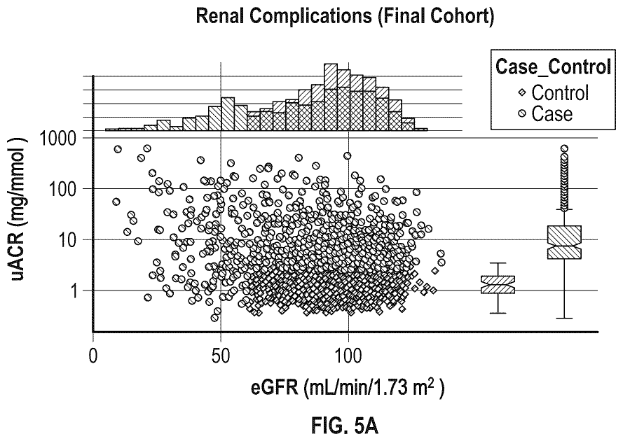

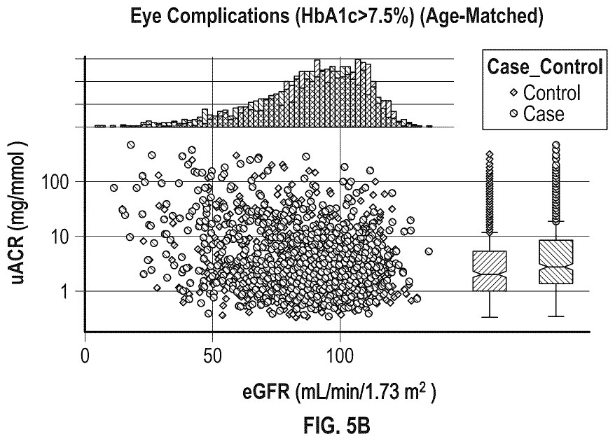

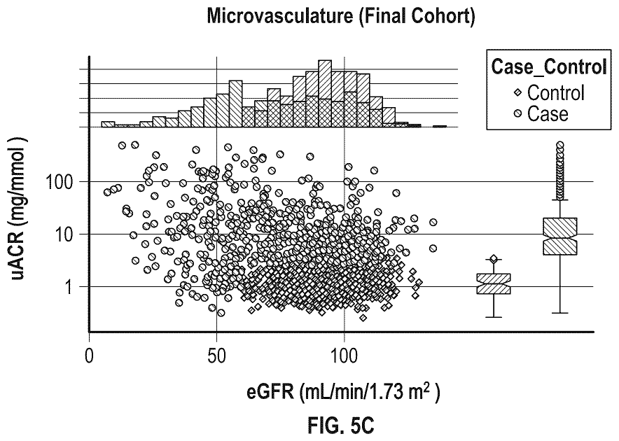

In the genetic data set controls were matched to cases based on age as a proxy of disease duration and gender was matched to ensure a demographic balance. The nephropathy genetic data set comprised 940 cases, and 940 controls. The retinopathy genetic data set comprised 1262 cases, and 1262 controls. The microvasculature data set comprised 696 cases and 696 controls. This rigorous design ensured the comparability of cases and controls, strengthening the ability of the method to identify genetic contributions to nephrovascular, retinal and microvasculature complications in T2D. FIGS. 5A-5C show the uACR and eGFR distributions of the cases and controls for the renal complication data set (FIG. 5A), the eye complications (retinopathy) data set (FIG. 5B), and the microvasculature complication data set (FIG. 5C).

The genetic data set was cleaned by filtering for quality control purposes (QC). Individuals with greater than 2% missing SNPs and SNPs with greater than 2% missing individuals were removed. SNPs with minor allele frequency below 5% were removed. Using a multidimensional scaling, individual genotypes were clustered in 2 dimensions. The cluster of individuals comprising individuals of European ancestry was identified and individuals that did not cluster with the European ancestry group were removed.

Genetic variant combinations were identified using variant interaction scores using the method described in Example 1. As in Example 1, the one or more genes associated with the genetic variant's combinations were identified. Gene grouping pairs were identified by selecting gene groupings containing the genes with significant variant interaction scores from the gene groupings described in Example 1. As in Example 1, SNPs not associated with genes in a pre-defined library of gene groupings were removed. The analysis resulted in about 200 interacting networks.

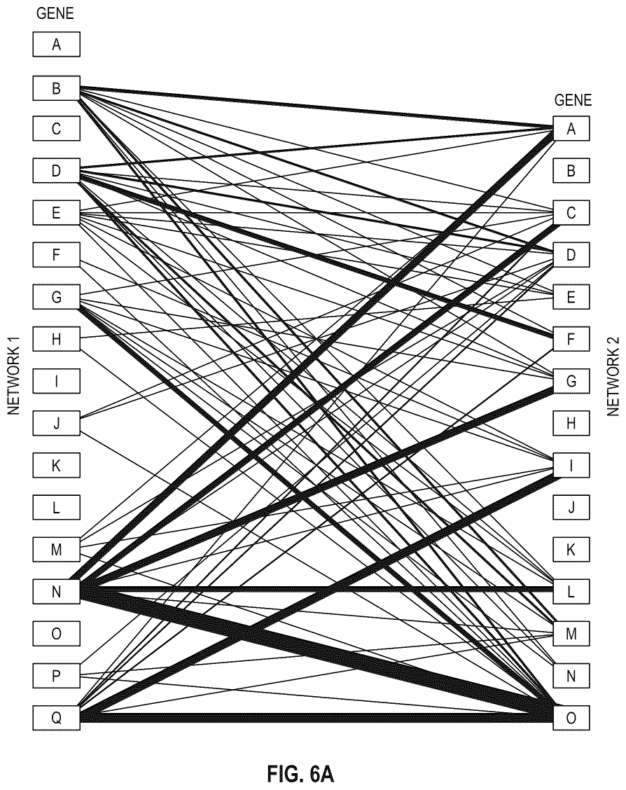

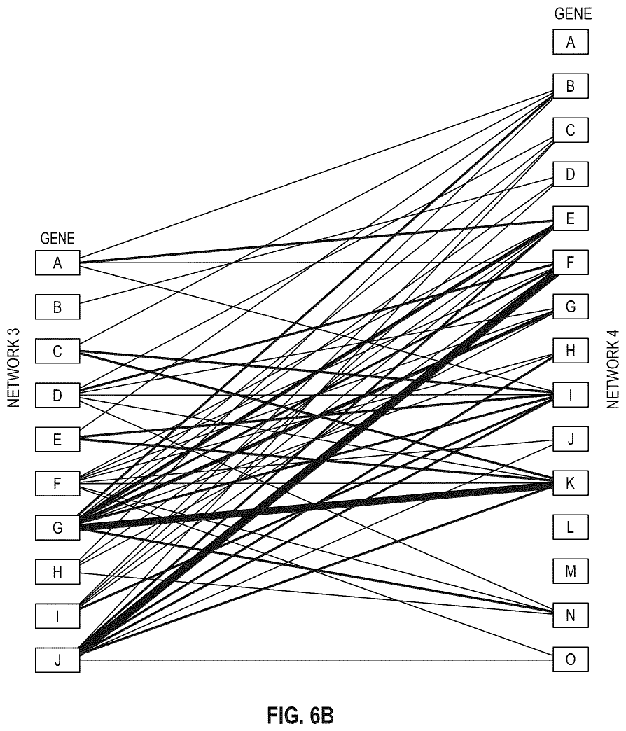

Interaction density scores were determined for each of the gene grouping pairs using the method described in Example 1. Two gene grouping pairs within the top 200 were selected for functional validation. The first gene grouping pair, shown in FIG. 6A, consisted of genes identified as part of Network 1 and Network 2. The second gene grouping pair, shown in FIG. 6B, consisted of genes identified as part of Network 3 and Network 4. The findings demonstrated simultaneous modulation of biology represented by grouping pairs (Network 1+Network 2) and (Network 3+Network 4), and associated gene pairs, yields synergistic efficacy in the disease-relevant assays. The relationship between the grouping pairs was not previously appreciated in the field. The methods described herein allows for the identification of grouping pairs and thus gene pairs whose synergistic effect on disease was not appreciated. As shown in FIG. 6A and FIG. 6B, the genes identified in each group as potential synergistic pairs are connected and the thickness of the connection in the figure relates to the variant interaction scores for SNPS associated with the gene.

### Viability Assay

A cell viability assay was designed to experimentally validate genes pairs in the identified gene grouping pairs as providing synergistic protection from glucotoxicity. HUV-EC-C cells were used as an endothelial cell model for diabetic microvascular complications. The cells were grown at 37° C., 5% CO₂, in Human Large Vessel Endothelial Cell Basal Medium with the addition of Large Vessel Endothelial Supplemented (LVES) and 8% heat inactivated FBS.

On day 1, cells were plated and transfected. 10,000 cells per well were reverse transfected in 96 well plates with siRNAs against single targets or in combination using 0.2 μl RNAiMAX and 25 nM total siRNA in OptiMem. For wells transfected with an siRNA targeting one gene in the pair, 12.5 nM targeting siRNA and 12.5 nM of a non-targeting siRNA were used. For the wells transfected with siRNA targeting both genes in the pair, 12.5 nM of each of the 2 siRNA targets were used. 10 μl of the transfection reagent were dispensed in each well, followed by 90 μl of the cell suspension containing about 10,000 cells, resulting in a final volume of 100 μl per well. A total of 5 replicates were prepared for each condition, two replicates were used as normal glucose plates (see below) and 3 replicates were used for the high glucose plates (see below).

On day 2, glucose treatments were performed, to model a T2D disease state. Cells were treated with 4 mM of glucose to model the normal state (control) or 50 mM to model a high glucose state. The high glucose solution was prepared as a 3X solution and 50 μl were added to the 100 μl of medium in the well. The normal glucose samples received 50 μl of normal glucose medium.

About 96 hours after the glucose addition, the cells were lysed and assessed for ATP as a surrogate for cell count using CellTiter-Glo™. The cell counts were normalized in two ways. First, the cell counts from each high glucose sample were normalized over the corresponding normal glucose sample. Second, the cell counts were normalized over the assay window of non-targeting siRNA transfected on each plate.

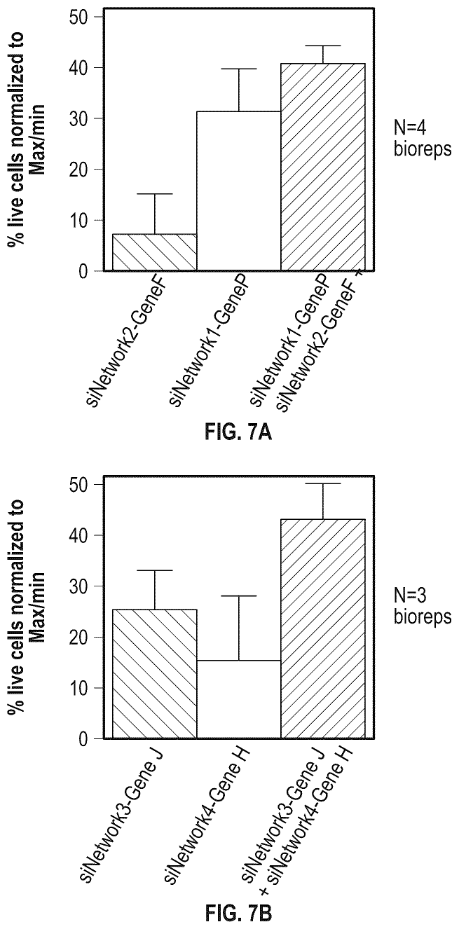

FIG. 7A and FIG. 7B show viability assay results at 4 days, normalized over the assay window for cells treated with high glucose conditions. In FIG. 7A cells treated with siRNAs directed to Network 2-Gene F and Network 1-Gene P had higher viability than cells treated with an siRNA directed to only one of the genes. In FIG. 7B cells treated with siRNAs directed to Network 3-Gene J and Network 4-Gene H had higher viability than cells treated with an siRNA directed to only one of the genes. Both duologs had similar protection from glucotoxicity in the 4-day viability assay. As described above, the combination of Network 2-Gene F and Network 1-Gene P and the combination of Network 3-Gene J, and Network 4-Gene were not previously known to have any functional relationship with each other in T2D, and the synergistic effect was not appreciated before these results. These results indicate synergistic protection from glucotoxicity for each duolog shown.

### Inflammation Assay

A cell inflammation assay was designed to experimentally validate gene pairs in the identified gene grouping pairs as protective from glucose-induced inflammation. HUV-EC-C cells were used as an endothelial cell model for diabetic microvascular complications. The cells were grown at 37° C., 5% CO₂, in Human Large Vessel Endothelial Cell Basal Medium with the addition of Large Vessel Endothelial Supplement (LVES) and 8% heat inactivated FBS.

On day 1, 10,000 cells per well were reverse transcribed using the same protocol as was used in the viability assay above. For the inflammation assay, samples were plated in duplicate for use in the normal and high glucose conditions. Cells were incubated with the transfection reagent at 37° C. for 5 hours. The medium was then changed to fresh complete medium to remove the transfection reagent.

On day 2, the cells were treated with normal and high glucose using the same procedure as in the viability assay above.

After about 48 hours in the high glucose condition, supernatant from each well was collected and the MCP1 monocyte chemoattractant protein 1 (MCP1) level was measured using an MCP-1 ELISA kit. MCP-1 is reported to be a measure of inflammatory response that is increased in diabetic vascular injury and diabetic retinopathy (Panee, Monocyte Chemoattractant Protein 1 (MCP-1) in obesity and diabetes, Cytokine 2012; Rubsam et al, Role of Inflammation in Diabetic Retinopathy, Int. J. Mol. Sci. 2018, 19(4), 942).

The supernatants were diluted 20-fold to ensure the concentrations fell within the range of the kit's included standards. MCP-1 concentrations in the medium were interpolated from the standard curve based on the optical density (OD) of each sample and corrected for the dilution factor. Cell viabilities were also collected using CellTiter-Glo to ensure proper interpretation of MCP-1 level.

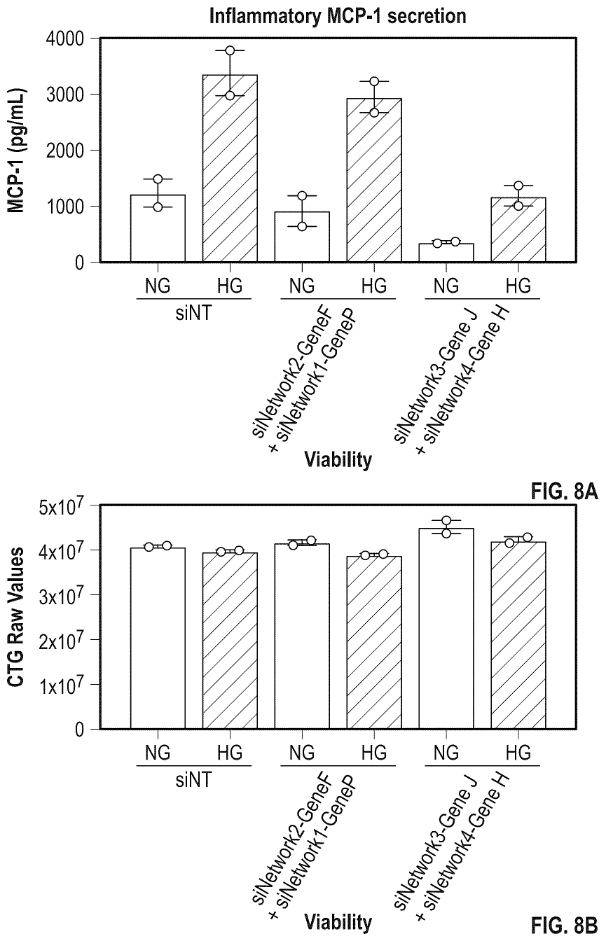

FIG. 8A shows MCP-1 levels for cells in the normal glucose (NG) or high glucose (HG) state upon treatment with non-target siRNAs (siNT) or the siRNAs targeting both genes in each of the duolog pairs; Network 3-Gene J+Network 4-Gene H knockdown showed protection from glucose-induced inflammation in the 2-day ELISA assay, whereas Network 1-Gene P+Network 2-Gene F knockdown showed no effect on glucose-induced inflammation. Cell viability (CTG Raw Values) was not affected by the two-day glucose treatment (FIG. 8B). Network 3-Gene J+Network 4-Gene H knockdown also reduced MCP-1 in normal glucose conditions compared to Network 2-Gene F+Network 1-Gene P knockdown, suggesting a role for this duolog in protection from inflammation. These results indicate that different phenotypes will yield distinct results across duologs and network pairs.

1. A method for identifying one or more gene combinations associated with a trait,
the method comprising:
identifying one or more genetic variant combinations from a genetic data set based on one or more variant interaction scores, wherein each variant interaction score is representative of an association between a trait and a joint state of all genetic variants of a single genetic loci combination of the one or more genetic loci combinations,
wherein the genetic data set comprises a plurality of inputs each having genetic information and at least one label indicative of the trait;
identifying one or more genes associated with genetic variants of the one or more identified genetic variant combinations;
selecting gene groupings from a library based on each gene grouping containing at least one of the identified one or more genes to form one or more gene grouping sets, wherein the library comprises groupings each representing an independent aspect of biology;
determining an interaction-density score for each of the one or more gene grouping sets wherein the interaction-density score is based on normalization of a grouping interaction score determined from the variant interaction scores for the genetic variant combinations between gene groupings in the gene grouping set; and
identifying the one or more gene combinations associated with the trait from at least one of the one or more gene grouping sets selected based on the determined interaction-density scores.

2. The method of claim 1, wherein the trait is the presence or absence of a human condition or is relevant to the human condition.

3. (canceled)

4. The method of claim 2, wherein the metric is assessed at the molecular, cellular, and/or organismal level.

5. The method of claim 2, wherein the human condition is a disease.

6. (canceled)

7. The method of claim 1, wherein genetic variant combinations contain genetic variants wherein each is independently selected from the group consisting of SNP, structural variation, copy number variation, insertion, deletion, translocation, and inversion.

8.-22. (canceled)

23. The method of claim 1, wherein the variant interaction score relates to a decisions rule comprising more than one statistical tests.

24. The method of claim 23, wherein the one or more statistical tests comprises a statistical test selected from a group consisting of a hypergeometric test, a logistic regression, or a linear regression.

25.-27. (canceled)

28. The method of claim 1, wherein the library comprising groupings each representing an independent aspect of biology comprises one or more gene networks.

29. The method of claim 1, wherein the library comprising groupings each representing an independent aspect of biology comprises gene groupings where each gene grouping has at least one non-overlapping gene when compared directly to another gene grouping in the library.

30. (canceled)

31. The method of claim 1, wherein the grouping interaction score is based on the variant interaction scores for the genetic variant combinations between the gene groupings in the gene grouping set.

32. The method of claim 1, wherein the group interaction score is normalized by dividing the score by the theoretical maximum of the number of genetic variant combinations between gene groupings in the gene grouping set.

33.-36. (canceled)

37. The method of claim 1, further comprising experimentally validating one or more of the identified gene combinations in a disease model system.

38. The method of claim 37, wherein an identified gene combination is validated based on an observed phenotype consistent with a phenotype that may treat or prevent a human condition.

39. (canceled)

40. The method of claim 37, wherein the disease model system comprises a cell assay, an organoid assay, or an animal model.

41. The method of claim 37, wherein the experimental validation comprises modulating an activity and/or expression level of an identified gene combination and comparing that to modulating an activity and/or expression level of one or more genes of the identified gene combination.

42.-43. (canceled)

44. The method of claim 1, further comprising selecting at least one of the identified gene combinations based on co-druggability.

45. The method of claim 1, further comprising experimentally validating the identified one or more gene combinations associated with the trait, wherein the identified one or more gene combinations comprises a gene combination comprising a first gene and a second gene.

46. The method of claim 45, wherein the experimental validating comprises performing a cell-based assay on:
(i) a first cell sample subjected to a programmable genome or transcriptome modulator to modulate the expression of the first gene;
(ii) a second cell sample subjected to a programmable genome or transcriptome modulator to modulate the expression of the second gene; and
(iii) a third cell sample subjected to the programmable genome or transcriptome modulator to modulate the expression of the first gene and the programmable genome or transcriptome modulator to modulate the expression of the second gene, and
determining if modulation of the expression of the first gene and the second gene results in a synergistic response observed in the cell-based assay.

47. The method of claim 46, wherein the first cell sample and the second cell sample are further subjected to a non-targeting programmable genome or transcriptome modulator control.

48.-50. (canceled)

51. The method of claim 45, wherein the experimental validating comprises performing a cell-based assay on:
(i) a first cell sample subjected to a drug moiety modulating the first gene, or an expression product thereof, and a non-target programmable genome or transcriptome modulator; and
(ii) a second cell sample subjected to a drug moiety modulating the first gene and a programmable genome or transcriptome modulator targeting the second gene, and
determining if there is an improved result in the cell-based assay from the second cell sample as compared to the first cell sample.

52.-56. (canceled)

57. A method of experimentally validating a gene combination comprising, the method comprising performing a cell-based assay on:
(i) a first cell sample subjected to a drug moiety modulating the first gene, or an expression product thereof, and a non-target programmable genome or transcriptome modulator; and
(ii) a second cell sample subjected to a drug moiety modulating the first gene and a programmable genome or transcriptome modulator targeting the second gene, and
determining if there is an improved result in the cell-based assay from the second cell sample as compared to the first cell sample.

58. The method of claim 57, wherein the gene combination is identified using a method according to claim 1.

59. A system for identifying one or more gene combinations associated with a trait, the system comprising:
one or more processors; and
memory storing one or more programs, the one or more programs configured to be executed by the one or more processors, the one or more programs including instructions for:
identifying one or more genetic variant combinations from a genetic data set based on one or more variant interaction scores, wherein each variant interaction score is representative of an association between a trait and a joint state of all genetic variants of a single genetic loci combination of the one or more genetic loci combinations,
wherein the genetic data set comprises a plurality of inputs each having genetic information and at least one label indicative of the trait;
identifying one or more genes associated with genetic variants of the one or more identified genetic variant combinations;
selecting gene groupings from a library based on each gene grouping containing at least one of the identified one or more genes to form one or more gene grouping sets,
wherein the library comprises groupings each representing an independent aspect of biology;
determining an interaction-density score for each of the one or more gene grouping sets wherein the interaction-density score is based on normalization of a grouping interaction score determined from the variant interaction scores for the genetic variant combinations between gene groupings in the gene grouping set; and
identifying the one or more gene combinations associated with the trait from at least one of the one or more gene grouping sets selected based on the determined interaction-density scores.

60. A method for identifying a subpopulation of patients for drug response, the method comprising:
identifying one or more genetic variant combinations from a genetic data set based on one or more variant interaction scores, wherein each variant interaction score is representative of an association between a trait and a joint state of all genetic variants of a single genetic loci combination of the one or more genetic loci combinations,
wherein the genetic data set comprises a plurality of inputs each having genetic information and at least one label indicative of the trait;
identifying one or more genes associated with genetic variants of the one or more identified genetic variant combinations;
selecting gene groupings from a library based on each gene grouping containing at least one of the identified one or more genes to form one or more gene grouping sets,
wherein the library comprises groupings each representing an independent aspect of biology;
determining an interaction-density score for each of the one or more gene grouping sets wherein the interaction-density score is based on normalization of a grouping interaction score determined from the variant interaction scores for the genetic variant combinations between gene groupings in the gene grouping set; and
identifying the one or more gene combinations associated with the trait from at least one of the one or more gene grouping sets selected based on the determined interaction-density scores;
wherein a gene in each of the one or more gene combinations is a target of the drug;
identifying a subpopulation of patients with genetic variation in one or more genes in the identified one or more gene combinations.
